(19) United States
(12) Patent Application Publication
(10) Pub. No.: US 2026/0319113 A1
(43) Pub. Date: Oct. 15, 2026

(54) Direct X-ray Detectors Based on Solution-Processed Amorphous Zero-Dimensional Organic Metal Halide Hybrid Films

(71) Applicant: The Florida State University Research Foundation, Inc., Tallahassee, FL (US)

(72) Inventors: Biwu Ma, Tallahassee, FL (US); Oluwadara Joshua Olasupo, Tallahassee, FL (US)

(21) Appl. No.: 19/637,290

(22) Filed: Apr. 2, 2026

Related U.S. Application Data
(60) Provisional application No. 63/783,528, filed on Apr. 4, 2025.

(51) Int. Cl. G01T 1/24; C07F 3/06

(52) U.S. Cl. CPC G01T 1/249; A61B 6/4208; A61B 6/4241; C07F 3/06; G01T 1/24

(57) ABSTRACT

Disclosed is a direct X-ray detector comprising: an amorphous organic metal halide hybrid (OMHH) material having a composition [R]_{z}(MₘXₙ)^{z-}, wherein (MₘXₙ)^{z-} is a metal halide anion, R⁺ is an organic cation, and wherein z, m, and n are integers; and at least one electrode in electrical communication with the OMHH material. Also disclosed are methods of using and making the same.

### CROSS-REFERENCE TO RELATED APPLICATIONS

This application claims the benefit of U.S. Provisional Application No. 63/783,528, filed Apr. 4, 2025, the entire contents of which are incorporated herein by reference.

### STATEMENT ACKNOWLEDGING GOVERNMENT SUPPORT

This invention was made with government support under grant no. DMR2204466 awarded by the National Science Foundation. The government has certain rights in the invention.

### TECHNICAL FIELD

The present disclosure relates to direct X-ray detectors based on solution-processed amorphous zero-dimensional organic-metal-halide hybrid films. In some aspects, the disclosure provides direct X-ray detectors comprising amorphous organic metal halide hybrid (OMHH) materials that exhibit high sensitivity, low detection limits, and stable performance for direct X-ray detection applications.

### BACKGROUND

X-ray detection is widely used in medical diagnostics, security screening, industrial inspection, and scientific research. Direct X-ray detectors are semiconductor devices that convert X-ray photons directly into electrical charges. Upon converting these charges into quantifiable electronic signals, direct X-ray detectors find various applications in photon-counting computed tomography (PCCT) for disease detection, radiography, real-time dosimetry for radiotherapy, and radiation quality assurance, as well as in industrial and security screening. Si, α-Se, and Cd(Zn)Te are the most commonly used semiconductors in modern direct X-ray detectors. While these inorganic semiconductors provide X-ray detectors with decent performance, numerous issues and challenges remain to be addressed to make them more affordable and improve their performance. Costly production, involving energy-intensive high-temperature processing, and a lack of structural flexibility are two major drawbacks of conventional inorganic semiconductors. To address these issues, researchers have been working on new types of semiconductors that offer low-cost preparation and high flexibility. One approach involves blending high-Z X-ray absorbers, such as inorganic nanoparticles (e.g., Bi₂O₃) and metal halide perovskites, with polymers to form large, flexible films. However, this approach has proved ineffective due to the non-uniformity of such blended films, evidenced by cracks and traps, resulting in poor detector performance.

To achieve large-size films with excellent uniformity for high-performance X-ray detectors, it is essential to develop material systems that combine high X-ray absorbers, such as metal oxides and halides, with organic materials in singular orientations, free from phase segregation.

These and other needs are addressed herein.

### SUMMARY

In some aspects, the present disclosure provides a direct X-ray detector comprising an amorphous organic metal halide hybrid (OMHH) material having a composition [R]_{z}(MₘXₙ)^{z-}, wherein (MₘXₙ)^{z-} is a metal halide anion, R⁺ is an organic cation, and wherein z, m, and n are integers; and at least one electrode in electrical communication with the OMHH material.

In some aspects, the present disclosure provides a method of detecting X-ray radiation comprising exposing an amorphous OMHH material to X-ray radiation, applying an electric field across the material, and detecting a signal generated by transported charge carriers.

In some aspects, the present disclosure provides a method of fabricating an X-ray detector comprising disposing a zero-dimensional amorphous OMHH film between a first electrode and a second electrode.

### BRIEF DESCRIPTION OF DRAWINGS

FIGS. 1A-1C demonstrate: (FIG. 1A) the schematic representation of molecular sensitization in 0D OMHHs, which metal halides (squares) act as X-ray absorbers and organic cations (L shape blocks) with lower bandgap as charge transporters; (FIG. 1B) from single crystalline 0D OMHHs to amorphous 0D OMHHs; and (FIG. 1C) the scheme showing the preparation of amorphous 0D (TPPCarz)₂ZnBr₄ films via facile solution processing and thermal annealing.

FIGS. 2A-2D provide schemes demonstrating the: (FIG. 2A) elemental mapping showing the presence of C, N, P, Zn, Br and their uniform distribution in a solution-processed 0D (TPPCarz)₂ZnBr₄ film; (FIG. 2B) scanning electron microscopy (SEM) image of a solution-processed 0D (TPPCarz)₂ZnBr₄ film; (FIG. 2C) PXRD pattern of a solution-processed 0D (TPPCarz)₂ZnBr₄ film; and (FIG. 2D) photoluminescence excitation (PLE) (emission at 405 nm) and photoluminescence (PL) emission (excited at 365 nm) spectra of solution-processed TPPCarzBr and 0D (TPPCarz)₂ZnBr₄ films.

FIGS. 3A-3D demonstrate the: (FIG. 3A) ultra-violet visible solid-state absorptance spectrum of solution processed 0D (TPPCarz)₂ZnBr₄ films with the inset showing an estimated bandgap of 3.0 eV; (FIG. 3B) current-voltage (I-V) plot showing the dark resistivity of a 1-mm thick film in a vertical device structure. The inset shows the schematic of the device structure; (FIG. 3C) dark I-V of a 0D (TPPCarz)₂ZnBr₄ film fitted under the space charge-limited current (SCLC) model; and (FIG. 3D) bias-dependent photoconductivity of a 0D (TPPCarz)₂ZnBr₄-based detector under continuous X-ray irradiation of 221.39 μGyₐᵢᵣs⁻¹.

FIGS. 4A-4D demonstrate the: (FIG. 4A) X-ray detection I-V plot at 20 V·mm⁻¹ electric field from dark to an X-ray dose rate of 221.39 μGyₐᵢᵣs⁻¹; (FIG. 4B) X-ray photocurrent density at various electric fields; (FIG. 4C) calculated X-ray sensitivity at different voltages/electric fields; and (FIG. 4D) signal-to-noise ratio (SNR) of 0D (TPPCarz)₂ZnBr₄ detectors versus various dose rates. The detection limit (LOD) of 6.01 nGyₐᵢᵣs⁻¹, estimated at 20 V·mm⁻¹, corresponds to the signal at an SNR of ~3.

FIGS. 5A-5C provide: (FIG. 5A) images of a 3-cm wide solution processed 0D (TPPCarz)₂ZnBr₄ film; (FIG. 5B) the X-ray detection I-V plot of a device based on 3-cm wide 0D (TPPCarz)₂ZnBr₄ film at 20 V·mm⁻¹ electric field from dark to an X-ray dose rate of 221.39 μGyₐᵢᵣs⁻¹ (inset shows the lateral device structure); and (FIG. 5C) X-ray photocurrent density versus dose rate with an estimated sensitivity of 1,802 μC Gyₐᵢᵣ⁻¹ cm⁻².

FIG. 6 provides schemes demonstrating the use of solution-processed amorphous zero-dimensional organic metal halide hybrid (0D (TPPCarz)₂ZnBr₄) films for direct X-ray detection. The amorphous films exhibit efficient molecular sensitization, with ZnBr₄²⁻ species serving as X-ray absorbers and TPPCarz⁺ moieties as charge transporters. The resulting detectors achieve a detection sensitivity of 2, 165 μC Gyₐᵢᵣ⁻¹ cm⁻² at 20 V·mm⁻¹ and a detection limit of 6.01 nGyₐᵢᵣ S⁻¹.

FIG. 7 provides a scheme demonstrating the synthesis of TPPCarzBr.

FIG. 8 provides the EDS spectrum of 0D (TPPCarz)₂ZnBr₄ film sample showing the percent weight composition of C, N, P, Zn, and Br.

FIG. 9 provides the frequency-dependent capacitance plot of 0D (TPPCarz)₂ZnBr₄ film with device architecture Au/(TPPCarz)₂ZnBr₄/Au shown in the inset.

FIGS. 10A-10B provide I-V plots showing the X-ray detection results of 0D (TPPCarz)₂ZnBr₄ from dark to a dose rate of 221.39 pGyₐᵢᵣs⁻¹ (FIG. 10A) at 5 V·mm⁻¹ and (FIG. 10B) at 10 V·mm⁻¹.

FIGS. 11A-11B provides schemes demonstrating: (FIG. 11A) the optical image of the detector fabricated using the 3 cm sample; and (FIG. 11B) the large-area X-ray detector.

FIGS. 12A-12B provide: FIG. 12A) the Raman spectrum of 0D (TPPCarz)₂ZnBr₄; and FIG. 12B) the time-resolved photoluminescence spectra for TPPCarzBr and 0D (TPPCarz)₂ZnBr₄. IRF represents the instrumental resolution function.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

In this specification and in the claims that follow, reference will be made to a number of terms, which shall be defined to have the following meanings:

As used herein, the terms “optional” or “optionally” mean that the subsequently described event or circumstance can or cannot occur and that the description includes instances where said event or circumstance occurs and instances where it does not.

It is appreciated that certain features of the disclosure, which are described for clarity in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” is used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The term “or” means “and/or.” Recitation of ranges of values is merely intended to serve as a shorthand method of referring individually to each separate value falling within the range unless otherwise indicated herein, and each separate value is incorporated into the specification as if it were individually recited herein. The endpoints of all ranges are included within the range and are independently combinable. All methods described herein can be performed in a suitable order unless otherwise indicated herein or otherwise clearly contradicted by context.

All disclosed values also include values that fall within +10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase ‘x, y, z, or greater’ should be interpreted to include the specific ranges of ‘x,’ ‘y,’ ‘z,’ ‘about x,’ ‘about y,’ and ‘about z’ as well as the ranges of ‘greater than x,’ greater than y,′ ‘greater than z,’ or ‘greater than about x,’ greater than about y,′ ‘greater than about z.’ In addition, the phrase “‘x’ to ‘y’,” where ‘x’ and ‘y’ are numerical values, also includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range.

Recitation of ranges of values herein is merely intended to serve as a shorthand method of referring individually to each separate value recited or falling within the range unless otherwise indicated herein, and each separate value is incorporated into the specification as if it were individually recited. Ranges provided herein are understood to be shorthand for all of the values within the range. For example, a range of 1 to 50 is understood to include any number, or combination of numbers, from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or sub-ranges from the group consisting of 10-40, 20-50, 5-35, etc. Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4).

As used herein, the term “composition” is intended to encompass a product comprising the specified ingredients in the specified amounts, as well as any product that results, directly or indirectly, from a combination of the specified ingredients in the specified amounts.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example embodiments.

As used herein, the term “substantially” means that the subsequently described event or circumstance completely occurs or that the subsequently described event or circumstance generally, typically, or approximately occurs.

Still further, the term “substantially” can, in some aspects, refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount. It is understood that this definition also includes the ranges when no word “about” is present.

In other aspects, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition or based on any other calculations as disclosed. It is understood that this definition also includes the ranges when no word “about” is present.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to. It is understood that this definition also includes the ranges when the word “about” is not present.

As used herein, the terms “substantially identical reference composition” and “substantially identical reference article” refer to a reference composition or article comprising substantially identical components in the absence of an inventive component. In another exemplary aspect, the term “substantially,” in, for example, the context “substantially identical reference composition” or “substantially identical reference article,” refers to a reference composition or an article comprising substantially identical components, and wherein an inventive component is absent or is substituted with a common in the art component.

By “contact” or other forms of the word, such as “contacted” or “contacting,” it is meant to add, combine, or mix two or more compounds, compositions, or materials under appropriate conditions to produce a desired product or effect. The term “react” is sometimes used when “contacting” results in a chemical reaction.

In certain aspects, the term “contact” refers to a physical interaction between two materials. As used herein, “contact” is to be interpreted broadly and encompasses situations in which the materials are chemically bonded, intermixed, fused, or merely in physical adjacency.

As used herein, the term “zero-dimensional” or “0D” refers to the structural dimensionality of a material where metal halide species are isolated from each other by organic cations, such that the metal halide anions do not form extended networks or continuous inorganic frameworks. In a zero-dimensional structure, the metal halide units exist as discrete molecular species surrounded by organic cations.

As used herein, the term “organic metal halide hybrid” or “OMHH” refers to a material comprising both organic cations and metal halide anions, where the organic and inorganic components are ionically bonded to form a hybrid structure. The OMHH may have various structural dimensionalities, including zero-dimensional (0D), one-dimensional (1D), two-dimensional (2D), or three-dimensional (3D) structures.

As used herein, the term “molecular sensitization” refers to a process in which metal halide species act as X-ray absorbers and organic cations with a lower bandgap act as charge transporters. In this process, X-ray radiation is absorbed by the metal halide anions, and the generated charge carriers are transported through the organic cation network.

As used herein, the term “direct X-ray detector” refers to a detector that directly converts X-ray radiation into electrical signals without the need for an intermediate scintillation step. In a direct X-ray detector, X-ray photons interact with the detector material to generate electron-hole pairs, which are then collected as an electrical signal.

As used herein, the term “amorphous” refers to a non-crystalline solid that lacks long-range atomic order. An amorphous material may be characterized by broad diffraction features in powder X-ray diffraction (PXRD) patterns, in contrast to the sharp diffraction peaks observed in crystalline materials.

As used herein, the term “bandgap” or “band gap” refers to the energy difference between the top of the valence band and the bottom of the conduction band in a semiconductor material, typically expressed in electron volts (eV). The bandgap determines the minimum energy required to excite an electron from the valence band to the conduction band.

As used herein, the term “light-emitting organic unit” refers to an organic moiety capable of emitting light upon excitation, typically comprising a conjugated aromatic or heteroaromatic system. In the context of this disclosure, the light-emitting organic unit may have a bandgap that enables charge transport in the OMHH material.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R) or(S) configuration. The compounds provided herein may either be enantiomerically pure or be diastereomeric or enantiomeric mixtures. It is to be understood that the chiral centers of the compounds provided herein may undergo epimerization in vivo. As such, one of ordinary skill in the art will recognize that administering a compound in its (R) form is equivalent, for compounds that undergo epimerization in vivo, to administering the compound in its(S) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Compounds described herein may contain one or more double bonds and, thus, potentially give rise to cis/trans (E/Z) isomers, as well as other conformational isomers unless stated to the contrary, all such possible isomers are contemplated, as well as mixtures of such isomers.

Compounds described herein may also present as an equilibrium of tautomers. For example, ketones with an α-hydrogen can exist in an equilibrium of the keto form and the enol form. Likewise, amides with an N-hydrogen can exist in an equilibrium of the amide form and the imidic acid form. Unless stated to the contrary, all possible tautomers of the compounds described herein are contemplated.

A dash (“-”) that is not between two letters or symbols is used to indicate a point of attachment for a substituent. For example, —(C═O)NH₂ is attached through the carbon of the keto (C═O) group.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., ═O), two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and/or can be formulated into a form with a shelf life of at least one month. A stable manufacturing intermediate or precursor to an active compound is stable if it does not degrade within the period needed for reaction or other use. A stable moiety or substituent group is one that does not degrade, react, or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: halo, nitro, cyano, azido, oxo, C₁-C₆ alkyl, C₁-C₆ haloalkyl, C₂-C₆ alkenyl, C₂-C₆ alkynyl, (C₃-C₇ cycloalkyl) (C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, A^{x}O—(C₀-C₆ alkyl)-, A^{x}S—(C₀-C₆ alkyl)-, (A^{x}A^{y}N)—(C₀-C₆ alkyl)-, A^{z}C(O)—(C₀-C₆ alkyl)-, A^{z}C(N)—(C₀-C₆ alkyl)-, and A^{z}S(O)—(C₀-C₆ alkyl)-, and A^{z}S(O)₂—(C₀-C₆ alkyl)-, wherein A^{x} and A^{y} are independently selected at each occurrence from A^{a}, A^{z}C(O)—, A^{z}C(N)—, A^{z}S(O)—, and A^{z}S(O)₂—, each of which may be optionally substituted with one or more B groups as allowed by valency; wherein A^{z} is independently selected at each occurrence from hydrogen, halo, C₁-C₀alkyl, C₁-C₆haloalkyl, C₂-C₀alkenyl, C₂-C₆alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OA^{a}, —SA^{a}, and -NA^{a}A^{b}, each of which may be optionally substituted with one or more B groups as allowed by valency; wherein Aa and Ab are independently selected at each occurrence from hydrogen, C₁-C₀alkyl, C₁-C₀haloalkyl, C₂-C₆alkenyl, C₂-C₀alkynyl, (C₃-C₇ cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted by one or more B groups as allowed by valency; and wherein B is independently selected at each occurrence from hydrogen, halo, nitro, cyano, azido, oxo, C₁-C₆ alkyl, C₁-C₆ haloalkyl, C₂-C₆ alkenyl, C₂-C₆ alkynyl, (C₃-C₇ cycloalkyl) (C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, (5- to 10-membered monocyclic or bicyclic heteroaryl)-(C₀-C₆ alkyl)-, A^{p}O—, A^{p}S—, A^{p}A^{q}N—, A^{o}C(O)—, A^{o}C(O)—O—, A^{o}C(O)-NA^{a}-, A^{o}S(O)₂—, A^{o}S(O)₂—O—, and A^{o}S(O)₂-NA^{a}-, wherein A^{o} is independently selected at each occurrence from AP, halo, A^{p}O—, and A^{p}A^{q}N—, and wherein AP and Ag are independently selected at each occurrence from hydrogen, C₁-C₆ alkyl, C₁-C₆ haloalkyl, C₂-C₆ alkenyl, C₂-C₆ alkynyl, (C₃-C₇ cycloalkyl) (C₀-C₃ alkyl)-, (3- to 8-membered monocyclic or bicyclic heterocycle)-(C₀-C₆ alkyl)-, (6- to 10-membered monocyclic or bicyclic aryl)-(C₀-C₆ alkyl)-, and (5- to 10-membered monocyclic or bicyclic heteroaryl)-(C₀-C₆ alkyl)-.

In certain aspects, when the disclosed compound has aryl rings, the following exemplary and unlimiting substitutions can be present. It is understood that R¹ and R², as shown in the formulas below, can be any Rs disclosed herein and are suitable for the desired application.

Monosubstituted Phenyl

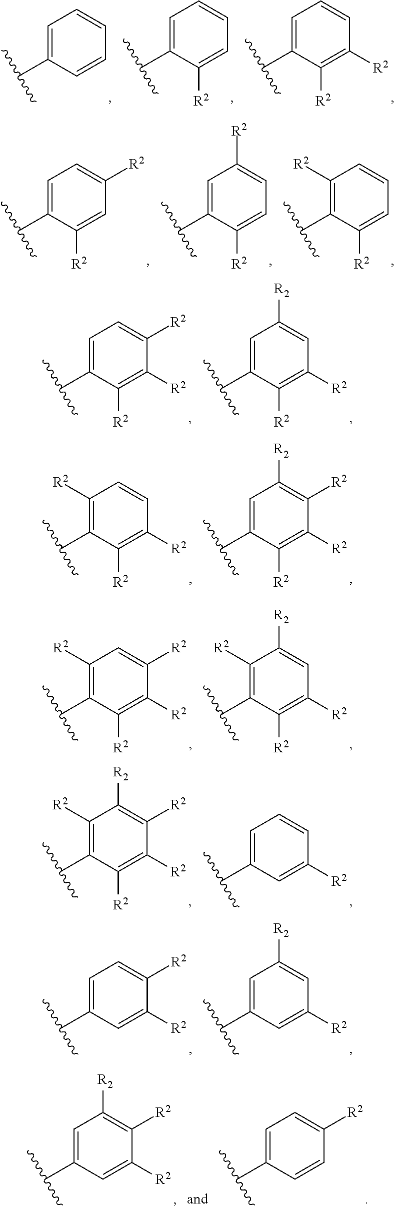

### Ortho Disubstituted Phenyl

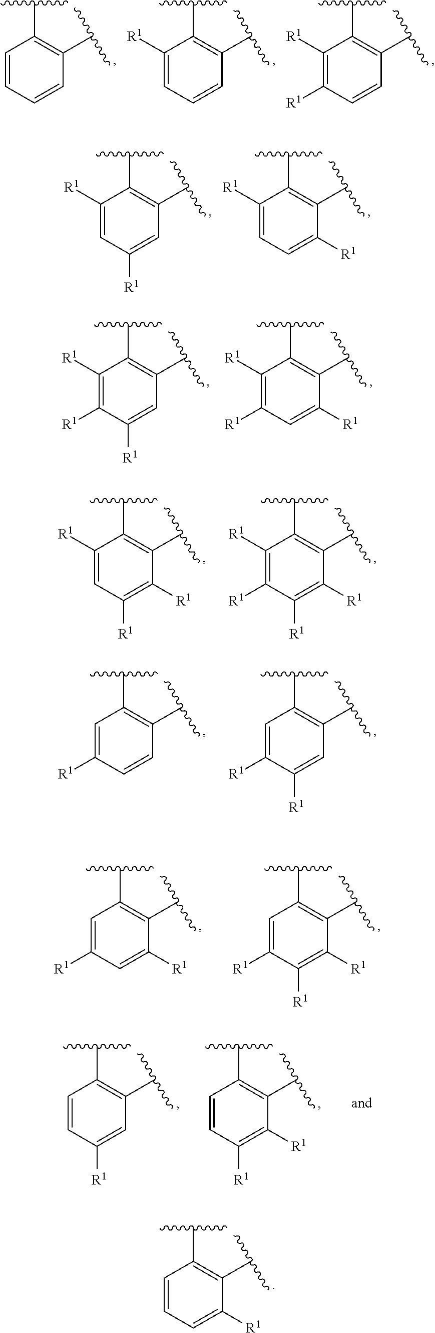

### Meta Disubstituted Phenyl

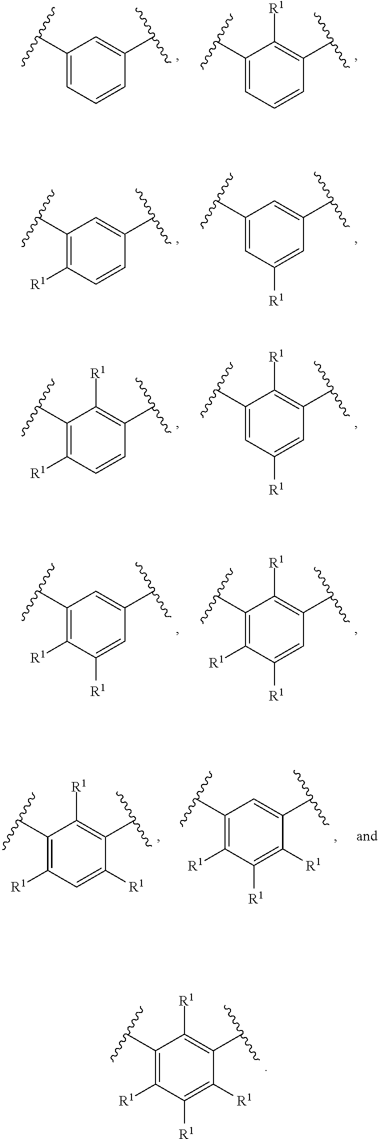

### Para Disubstituted Phenyl

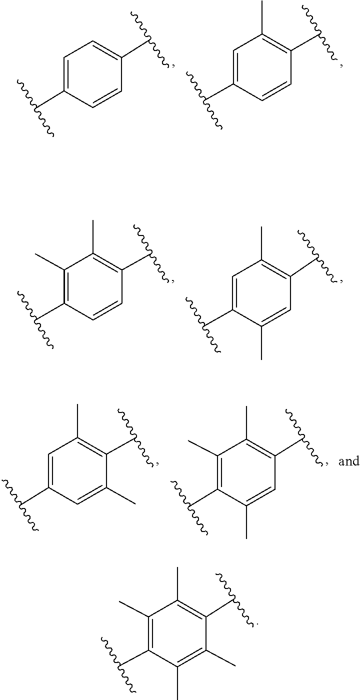

### 2-Substituted 5-Membered Aryl

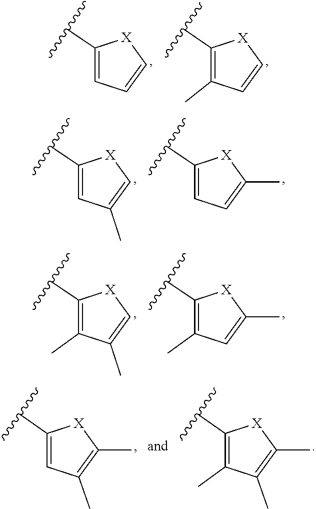

### 3-Substituted 5-Membered Aryl

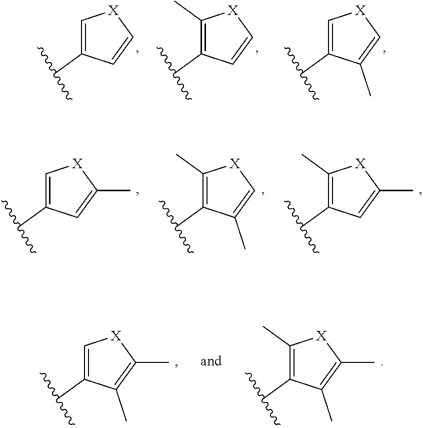

2,3-Disubstituted 5-Membered Aryl

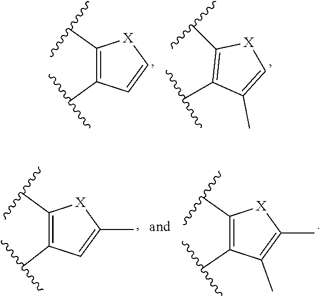

3,4-Disubstituted 5-Membered Aryl

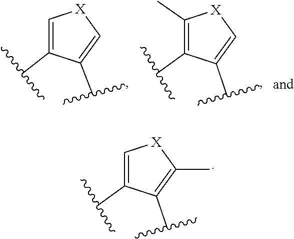

1,3,4-trisubstituted Phenyl

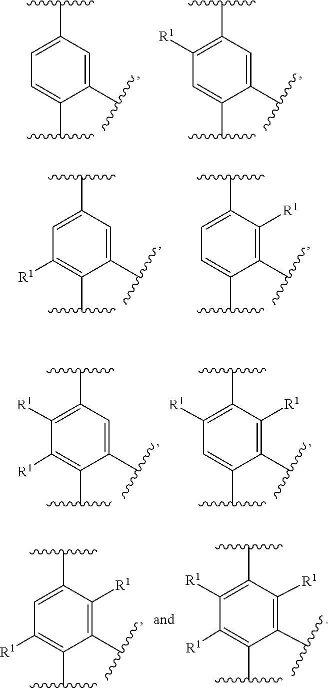

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person in the context in which said functional groups are recited.

As used herein, the symbol

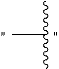

(which hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

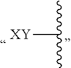

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

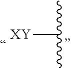

infers that when R³ is “XY”, the point of attachment bond is the same bond as the bond by which R³ is depicted as being bonded to CH₃.

“Halo” or “halogen” independently indicates any fluoro, chloro, bromo or iodo.

The term “nitro,” as used herein, is represented by the formula —NO₂.

The term “cyano,” as used herein, is represented by the formula —CN

The term “azido,” as used herein, is represented by the formula —N₃.

The term “oxo,” as used herein, is represented by the formula ═O.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with the length of each member of the range described as an independent species. For example, C₁-C₀alkyl, as used herein, indicates an alkyl group having 1, 2, 3, 4, 5, or 6 carbon atoms and is intended to mean that each of these is described as an independent species, and C₁-C₄alkyl, as used herein, indicates an alkyl group having 1, 2, 3, or 4 carbon atoms and is intended to mean that each of these is described as an independent species. When C₀-Cₙalkyl is used herein in conjunction with another group, for example (C₃-C₇ cycloalkyl) C₀-C₄alkyl, or-C₀-C₄ (C₃-C₇ cycloalkyl), the indicated group, in this case cycloalkyl, is either directly bound by a single covalent bond (Coalkyl), or attached by an alkyl chain, in this case 1, 2, 3, or 4 carbon atoms. Alkyls can also be attached via other groups such as heteroatoms, such as—O—C₀-C₄alkyl(C₃-C₇ cycloalkyl). Examples of alkyl include but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some aspects, the alkyl group is optionally substituted as described herein.

“Haloalkyl” refers to an alkyl group that is substituted with one or more halo groups, e.g., fluoro, chloro, bromo, iodo, or combinations thereof.

“Cycloalkyl” is a saturated or partially unsaturated mono- or multicyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some aspects, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₀alkenyl (i.e., having 2, 3, 4, 5, or 6 carbons). The specified ranges as used herein indicate an alkenyl group, with each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₀alkynyl (i.e., having 2, 3, 4, 5, or 6 carbons). The specified ranges as used herein indicate an alkynyl group, with each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include the fusion to a 4- to 7- or 5- to 7-membered saturated or partially unsaturated cyclic group that optionally contains 1, 2, or 3 heteroatoms independently selected from N, O, B, P, Si, and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing-O—O—, —O—S—, and —S—S-portions. Examples of saturated heterocycle groups, including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited to, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical, and the point of attachment is the heterocycle ring. Bicyclic heterocycle also includes heterocyclic radicals that are fused with a carbocyclic radical. Representative examples include but are not limited to, partially unsaturated condensed heterocyclic groups containing 1 to 5 nitrogen atoms, for example, indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms. In one aspect, the heterocycle group is optionally substituted as described herein.

“Heteroaryl” refers to a stable monocyclic, bicyclic, or multicyclic aromatic ring that contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. In one aspect, the heteroaryl group is optionally substituted as described herein.

As used herein, the term “derivative” refers to a compound having a structure derived from the structure of a parent compound (e.g., a compound disclosed herein) and whose structure is sufficiently similar to those disclosed herein and based upon that similarity would be expected by one skilled in the art to exhibit the same or similar activities and utilities as the claimed compounds, or to induce, as a precursor, the same or similar activities and utilities as the claimed compound. Exemplary derivatives include but are not limited to, salts, esters, amides, salts of esters or amides, and N-oxides of a parent compound.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers, such as Sigma-Aldrich (formerly MilliporeSigma, Burlington, MA) or Thermo Fisher Scientific Inc. (Waltham, MA), or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8th Edition); and Larock's Comprehensive Organic Transformations (John Wiley and Sons, 3rd edition, 2017).

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Direct X-Ray Detector Compositions and Structures

In various aspects, the present disclosure provides a direct X-ray detector comprising an amorphous organic metal halide hybrid (OMHH) material having a composition [R]_{z}(MₘXₙ)^{z-}, wherein (MₘXₙ)^{z-} is a metal halide anion, R⁺ is an organic cation, and wherein z, m, and n are integers. The direct X-ray detector further comprises at least one electrode in electrical communication with the OMHH material.

In some aspects, z may be 1 to 6, 1 to 5, 1 to 4, 1 to 3, 2 to 4, or 2. In some aspects, m can be 1 to 12, 1 to 10, 1 to 8, 1 to 6, 1 to 4, 1 to 2, or 1. In some aspects, n can be 1 to 24, 1 to 20, 1 to 16, 1 to 12, 1 to 8, 1 to 6, or 4. In certain aspects, z is 2, m is 1, and n is 4, such that the metal halide anion has the formula (MX₄)²⁻.

In various aspects, M may be a metal selected from Mn, Ni, Cu, Zn, Pd, Ag, Sn, Sb, Ti, Pb, Bi, and combinations thereof. In some aspects, M is Zn. In various aspects, X may be a halide chosen from Cl, Br, I, and combinations thereof. In some aspects, X is Br.

In some aspects, the OMHH may be in the form of a thin film. In various aspects, the film is a continuous film. In still further aspects, the film can have any dimensions that are required for the desired size of the detector. The amorphous nature of the OMHH film may be confirmed by powder X-ray diffraction (PXRD), which may display broad diffraction features in contrast to the sharp diffraction peaks observed in crystalline materials.

### Organic Cation Compositions

In various aspects, the organic cation R⁺ may comprise a phosphonium cation having a structure

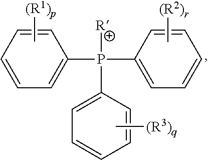

wherein R′ is a substituted or unsubstituted light-emitting organic unit covalently bound to phosphorus. In some aspects, R¹, R², and R³ may independently be selected at each occurrence from F, Cl, Br, I, NO₂, CN, COOH, C₁-8alkyl, C₁₋₈heteroalkyl, C₆₋₁₄aryl, C₃₋₈cycloalkyl, 3- to 8-membered heterocyclyl, and 5- to 8-membered heteroaryl. In some aspects, p, q, and r may independently be integers from 0 to 5, including 1, 2, 3, or 4.

In some aspects, the light-emitting organic unit R′ may comprise carbazole, acridine, phenoxazine, phenothiazine, fluorene, pyrene, anthracene, naphthalene, or derivatives thereof. In some aspects, R′ may comprise

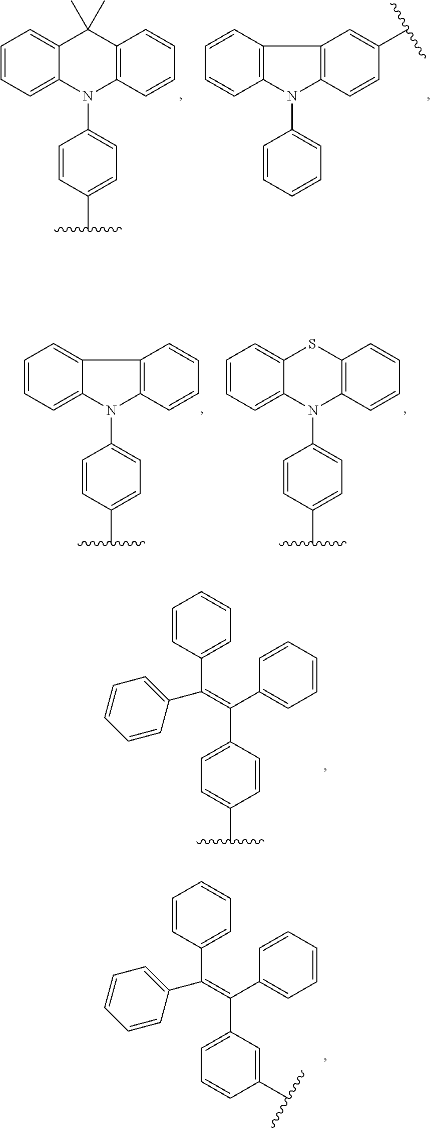

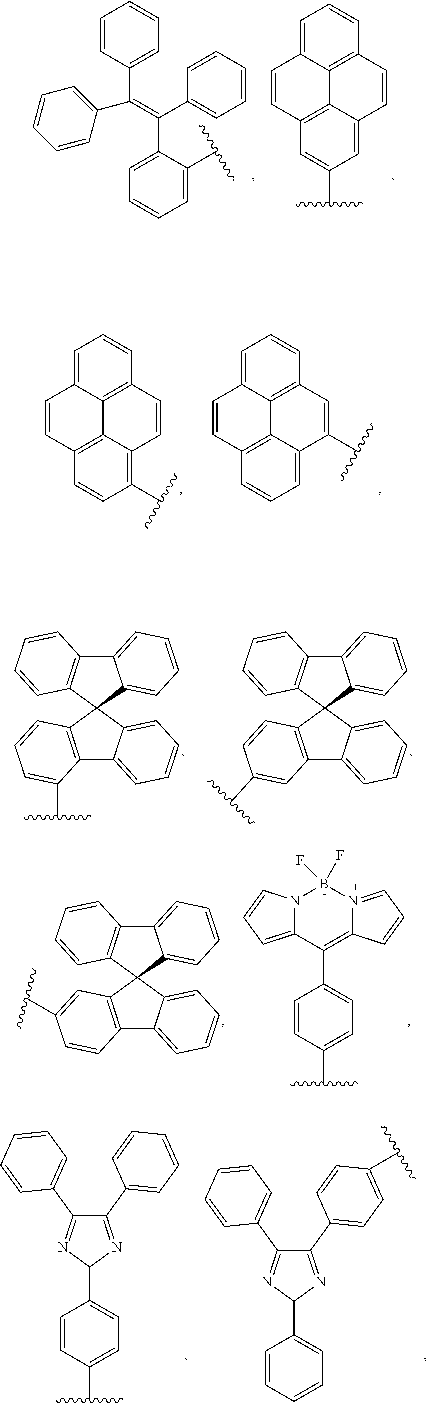

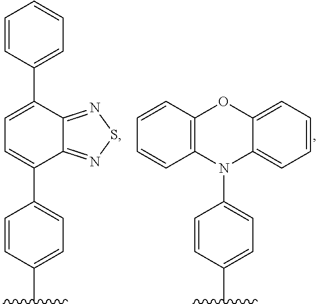

or a combination thereof.

In various aspects, R⁺ may be selected from triphenyl (9-phenyl-9H-carbazol-3-yl)phosphonium (TPPCarz⁺), (4-(9,9-dimethylacridin-10 (9H)-yl)phenyl) triphenylphosphonium (DMAC-TPP⁺), (4-(10H-phenoxazin-10-yl)phenyltriphenylphosphonium (PXZ-TPP⁺), or any combination thereof.

In some aspects, R⁺ may have a bandgap that is lower than or equal to the bandgap of (MₘXₙ)^{z-} such that the metal halide anions are configured to absorb X-ray radiation primarily, and the R⁺ is configured to transport charge carriers generated upon absorption of the X-ray radiation. This arrangement may enable efficient molecular sensitization of the organic cations by the metal halide anions.

In various aspects, the OMHH may be (TPPCarz)₂ZnBr₄, (DMAC-TPP)₂ZnBr₄, (PXZ-TPP)₂ZnBr₄, or combinations thereof.

In some aspects, the organic cation R⁺ may comprise other phosphonium cations with light-emitting organic units. In various aspects, R⁺ may comprise phenothiazine-based phosphonium cations such as (4-(phenothiazin-10H-yl)phenyl) triphenylphosphonium, fluorene-based phosphonium cations, pyrene-based phosphonium cations, anthracene-based phosphonium cations, naphthalene-based phosphonium cations, or other carbazole derivatives with different N-substituents.

In various aspects, the metal halide anion (MₘXₙ)^{z-} may comprise ZnCl₄²⁻, ZnBr₄²⁻, Znl₄²⁻, CuBr₄²⁻, CuCl₄²⁻, NiBr₄²⁻, PdBr₄²⁻, AgBr₄³⁻, SnBr₆⁴⁻, Snl₆⁴⁻, PbCl₄²⁻, Pbl₄²⁻, PbBr₄²⁻, BiBr₆³⁻, BiI₆³⁻, BiCl₆³⁻, or combinations thereof.

### Performance Parameters

In various aspects, the OMHH may exhibit a bulk resistivity greater than 10⁸ Ω·cm, or greater than 10⁹ Ω·cm, or greater than 5×10⁹ Ω·cm, or greater than 10¹⁰ Ω·cm, or at least 10¹⁰ Ω·cm, or at least 1.5×10¹⁰ Ω·cm. In some aspects, the OMHH may exhibit a bulk resistivity of about 1.82×10¹⁰ Ω·cm. The high bulk resistivity may be attributed to the zero-dimensional structure, which has low intrinsic carrier concentrations that may effectively suppress dark current.

In various aspects, the OMHH may exhibit a trap density of less than 1013 cm⁻³, or less than 10¹² cm⁻³, or less than 5×10¹¹ cm⁻³, or less than 10¹¹ cm⁻³, or less than 8×10¹⁰ cm⁻³. In some aspects, the OMHH may exhibit a trap density of about 7.04×10¹⁰ cm⁻³. The low trap density may be advantageous for minimizing charge carrier recombination and enhancing carrier extraction efficiency.

In various aspects, the OMHH may exhibit a charge carrier mobility-lifetime product (UT) of at least about 10⁻⁶ cm²·V⁻¹, or at least about 10⁻⁵ cm²·V⁻¹, or at least about 5×10⁻⁵ cm²·V⁻¹, or at least about 10⁻⁴ cm²·V⁻¹, or at least about 2×10⁻⁴ cm²·V⁻¹, or at least about 4×10⁻⁴ cm²·V⁻¹. In some aspects, the OMHH may exhibit a charge carrier mobility-lifetime product of about 4.63×10⁻⁴ cm²·V⁻¹. The high mobility-lifetime product may be attributed to the presence of charge-conducting organic cations.

In various aspects, the direct X-ray detector may exhibit a sensitivity of at least about 100 μC Gyₐᵢᵣ⁻¹ cm⁻², or at least about 250 μC Gyₐᵢᵣ⁻¹ cm⁻², or at least about 500 μC Gyₐᵢᵣ⁻¹ cm⁻², or at least about 750 μC Gyₐᵢᵣ⁻¹ cm⁻², or at least about 1,000 μC Gyₐᵢᵣ⁻¹ cm⁻², or at least about 1,250 μC Gyₐᵢᵣ⁻¹ cm⁻², or at least about 1,500 μC Gyₐᵢᵣ⁻¹ cm⁻², or at least about 1,750 μC Gyₐᵢᵣ⁻¹ cm⁻², or at least about 2,000 μC Gyₐᵢᵣ⁻¹ cm⁻² at 20 V·mm⁻¹. In some aspects, the direct X-ray detector may exhibit a sensitivity of about 2, 165 μC Gyₐᵢᵣ⁻¹ cm⁻² at 20 V·mm⁻¹.

In various aspects, the direct X-ray detector may have a detection limit of less than or equal to 500 nGyₐᵢᵣs⁻¹, or less than or equal to 250 nGyₐᵢᵣ s⁻¹, or less than or equal to 100 nGyₐᵢᵣs⁻¹, or less than or equal to 50 nGyₐᵢᵣ s⁻¹, or less than or equal to 25 nGyₐᵢᵣs⁻¹, or less than or equal to 10 nGyₐᵢᵣs⁻¹, or less than or equal to 8 nGyₐᵢᵣs⁻¹ at 20 V·mm⁻¹. In some aspects, the direct X-ray detector may have a detection limit of about 6.01 nGyₐᵢᵣ s⁻¹ at 20 V·mm⁻¹.

In some aspects, ionic migration may be substantially suppressed in the direct X-ray detector. Without wishing to be bound by any theory, the suppression of ionic migration may be attributed to the zero-dimensional structure, where bulky organic cations introduce steric effects that increase the activation energy barrier for ionic migration.

In various aspects, the direct X-ray detector may comprise a first electrode and a second electrode, wherein the amorphous OMHH material is disposed between the first and the second electrodes. The electrodes may be configured in a vertical device structure or a lateral device structure.

In some aspects, the first and the second electrodes may comprise Au, Ag, indium tin oxide (ITO), carbon nanotubes (CNT), Cu, Ni, Al, or combinations thereof. In some aspects, the electrodes may comprise silver paint electrodes.

In various aspects, the detector may have at least one lateral dimension that is 0.1 to 100 cm, or 0.5 to 75 cm, or 1 to 50 cm, or 1 to 25 cm, or 1 to 10 cm, or 2 to 5 cm, or about 3 cm. In some aspects, the at least one lateral dimension may be a diameter or a width of the detector. The solution-processing method may enable fabrication of large-area detectors.

In various aspects, the OMHH film may have a thickness of 0.01 mm to 50 mm, or 0.05 mm to 25 mm, or 0.1 mm to 10 mm, or 0.25 mm to 5 mm, or 0.5 mm to 2 mm, or about 1 mm. The thickness may be adjusted by varying the precursor solution concentration, amount, and mold dimensions.

In some aspects, the detector may exhibit stable performance with a reduction of less than 5%, or less than 2% in measured photocurrent at X-ray dose rates up to 221.39 μGyₐᵢᵣs⁻¹ at 20 V·mm⁻¹ for 12 h, or 24 h, or 48 h.

### METHODS

In various aspects, the amorphous OMHH material may be formed by a solution-based process. The solution-based process may comprise preparing a precursor solution comprising an organic halide salt and a metal halide salt dissolved in a solvent, depositing the precursor solution, and annealing the deposited precursor solution.

In some aspects, the solvent may comprise dimethylformamide (DMF), dimethyl sulfoxide (DMSO), gamma-butyrolactone (GBL), or combinations thereof. In some aspects, the organic halide salt and the metal halide salt may be dissolved in a molar ratio of about 2:1.

In some aspects, the precursor solution may be deposited by drop-casting, spin-coating, blade-coating, slot-die coating, or other solution deposition methods. In some aspects, the precursor solution may be deposited into a mold to control the shape and dimensions of the resulting film.

In various aspects, the amorphous OMHH material may be formed at a temperature of less than 300° C., or less than 250° C., or less than 200° C., or less than 175° C., or about 150° C. In some aspects, the annealing may be performed for 6 hours to 96 hours, or 12 hours to 72 hours, or 24 hours to 60 hours, or about 48 hours, to remove the solvent and obtain solvent-free film samples.

In various aspects, the present disclosure provides a method of detecting X-ray radiation. The method may comprise exposing an amorphous organic metal halide hybrid (OMHH) material having a composition [R]_{z}(MₘXₙ)^{z-} to X-ray radiation, wherein the metal halide anions are configured to absorb X-ray radiation and the R⁺ is configured to transport charge carriers generated upon absorption of the X-ray radiation.

The method may further comprise applying an electric field across the material. In some aspects, the electric field may be about 1 V·mm⁻¹ to about 100 V·mm⁻¹, or about 5 V·mm⁻¹ to about 50 V·mm⁻¹, or about 10 V·mm⁻¹ to about 30 V·mm⁻¹, or about 15 V·mm⁻¹ to about 25 V·mm⁻¹, or about 20 V·mm⁻¹.

The method may further comprise detecting a signal generated by the transported charge carriers. The signal may be a photocurrent that increases with increasing X-ray dose rate. The method may be performed in ambient air.

### Examples

The following examples are put forth so as to provide those of ordinary skill in the art with a complete disclosure and description of how the compounds, compositions, articles, devices, and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is degrees C. or is at ambient temperature, and pressure is at or near atmospheric.

Zero-dimensional (0D) organic metal halide hybrids (OMHHs), which incorporate high X-ray-absorbing metal halides and semiconducting organic cations via ionic bonding, are highly promising candidates for this purpose. Previously the inventors have demonstrated direct X-ray detectors based on single crystalline 0D OMHH (TPA-P)₂ZnBr₄ (TPA-P⁺=4-(4-(diphenylamino)phenyl)-1-propylpyridin-1-ium) with molecular sensitization, in which metal halide species (ZnBr₄²⁻) act as X-ray absorbers and organic semiconducting cations (TPA-P⁺) as charge transporters. Molecular sensitization in 0D OMHHs offers unique design and engineering opportunities, enabling the rational selection of organic species with desired intrinsic properties, such as electrical conductivity and flexibility. However, the scalability of 0D OMHH single crystals can be limited due to the time-intensive nature of the crystal growth and the challenge of producing large crystals exceeding a few centimeters. Solution-grown amorphous films present an interesting opportunity for manufacturing of direct X-ray detectors on a large scale.

Here, the development of solution-processed amorphous 0D OMHH films molecularly sensitized for efficient direct X-ray detection is reported. By reacting non-crystalline organic halide salt, TPPCarzBr, with wide-bandgap metal halide, ZnBr₂, a large-area, uniform, and amorphous 0D OMHH, (TPPCarz)₂ZnBr₄ films were fabricated to form a detector. Similar to single-crystalline 0D (TPA-P)₂ZnBr₄, the amorphous 0D (TPPCarz)₂ZnBr₄ exhibits highly efficient molecular sensitization, where ZnBr₄²⁻ anions serve as X-ray absorbers and TPPCarz⁺ cations function as charge transporters. Direct X-ray detectors based on amorphous 0D (TPPCarz)₂ZnBr₄ films have been successfully fabricated and tested as described herein. As it is shown below, at an electric field of 20 V·mm⁻¹, these detectors exhibit a high resistivity of 1.82×10¹⁰ Ω·cm, an impressive sensitivity of 2, 165 μC Gyₐᵢᵣ⁻¹ cm⁻², and a very low detection limit of 6.01 nGyₐᵢᵣ s⁻¹, achieving performance comparable to conventional inorganic semiconductor-based devices. Additionally, a large-area detector (~3 cm in diameter) was fabricated using 0D (TPPCarz)₂ZnBr₄ films, demonstrating a record high sensitivity of 1,802 μC Gyₐᵢᵣ⁻¹ cm⁻² for large-area solution-processed detectors.

### EXAMPLE 1-Materials and Methods

Chemicals: Triphenylphosphine (TPP), Aldrich, 99%), Zinc (II) bromide, 3-bromo-9-phenylcarbazole, nickel (II) bromide, tetraphenylphosphonium bromide (97%), diethyl ether (Et₂O, anhydrous), and dimethylformamide (DMF, anhydrous) were purchased from Sigma-Aldrich. Ethylene glycol was purchased from VWR. All reagents and solvents were used without further purification unless otherwise stated.

Synthesis of Triphenyl (9-Phenyl-9H-Carbazol-3-YI) Phosphonium Bromide (TPPCarzBr): TPPCarzBr was synthesized by following previously reported methods. Here, 655 mg (2.5 mmol) of triphenylphosphine, 805 mg (2.5 mmol) of 3-bromo-9-phenylcarbazole, and 62.5 mg (0.25 mmol) of NiBr₂ were added into 12.5 mL of ethylene glycol. The mixture was stirred at 150° C. for 5 hours. The resulting deep green solution was then dissolved in water and extracted with a dichloromethane solvent. Subsequently, the resulting white powder product was washed with diethyl ether, obtaining a yield of ~ 90%.

Preparation of (TPPCarz)₂ZnBr₄ Film: 2 mmol of TPPCarzBr (1.168g) and 1 mmol of ZnBr₂ (225.198 mg) were dissolved in 1 mL of DMF until a clear precursor solution was obtained. The subsequent precursor solution was filtered using a 0.22 μm PTFE filter and then carefully drop-cast into a resin mold. The sample was carefully annealed at ~150° C. in a fume hood for two days to obtain solvent-free film samples. Large-area films were prepared the same way.

Optical Characterization: Ultraviolet-visible solid-state absorption was measured at room temperature using an Edinburgh FS5 spectrometer coupled with an Edinburgh SC-30 integrating sphere. A 150 W Xe lamp was used as the light source, and absorption was measured in the wavelength range of 300 to 700 nm. Steady-state photoluminescence (PL) measurements were performed using the same instrument. Time-Correlated Single Photon Counting (TCSPC) was performed for 10000 counts using excitation from an Edinburgh EPL-360 picosecond pulsed diode laser. The PL decay was fitted using a biexponential decay function for a TPPCarzBr and 0D (TPPCarz)₂ZnBr₄. The weighted average lifetime, Tavg was computed according to Equation (1) below:

$${τ}_{avg} = \frac{\sum {α}_{i} {τ}_{i}^{2}}{\sum {α}_{i} {τ}_{i}} \qquad ( 1 )$$

Powder XRD characterization: Powder XRD patterns of (TPPCarz)₂ZnBr₄ were obtained using a Rigaku SmartLab diffractometer having a Cu Kα as the radiation source (where)=1.542 Å). At room temperature, the diffractometer's X-ray tube operated at 40 KV and 44 mA while recording XRD patterns ranging from 5° to 50° with a step size of 0.01°.

Morphological and Elemental Characterization: The Nova NanoSEM 400 (FEI Company) captured SEM Images and corresponding EDS spectra of individual components at a scanning voltage of 5.0 kV.

Raman Spectra Characterization. Measurements on the samples were performed using a Horiba LabRAM HR Evolution spectrometer, employing a 785 nm laser, 600 gr/mm grating, and a 50× objective. The spectra were recorded over the 100-3200 cm⁻¹ range with laser power reduced to <0.5 mW to prevent thermal effects.

Device Fabrication and Electrical Characterization: All devices were completed using silver paint electrodes, while platinum and copper wires were used to connect them to the external circuitry. Current-voltage characteristics were measured using a PV Measurements semiconductor characterization workstation. The electrochemical impedance measurement was performed using a Biologic SP300 electrochemical analyzer within a frequency range of 1 Hz to 7 MHz. A Keithley 2400 SourceMeter was employed to apply different bias voltages simultaneously and measure the corresponding current responses throughout the experiments. For the X-ray detection measurements, a commercially available MOXTEX X-ray tube was secured with a tungsten anode target and a maximum power of 4 W.

Limit-of-Detection Calculation and Dose Rates Measurements. The detector's dark current, I_{dark} as well as the average dark current, Avg_{dark} were recorded multiple times in a dark room. Dose rates were carefully regulated by varying the tube's current and voltage, and the corresponding X-ray doses were recorded using a calibrated handheld dosimeter. By tuning to low X-ray tube current and voltage values, corresponding X-ray dose rates from 1.8 nGyₐᵢᵣs⁻¹ to 60 nGyₐᵢᵣs⁻¹ were successfully recorded.

Signal-to-Noise Ratio (SNR) Calculation for Limit of Detection (LOD) Estimation: According to The International Union of Pure and Applied Chemists (IUPAC), the LOD is estimated as the dose rate where the SNR is approximately equal to 3, where:

$$S N R = \frac{{I}_{signal}}{{I}_{noise}} \qquad ( 2 )$$

$${I}_{signal} = {Avg}_{Xray} - {Avg}_{dark} \qquad ( 3 )$$

Where Avg_{Xray} is the average of the X-ray currents at a particular dose rate and Avgdark is the average of the dark currents. Iₙₒᵢₛₑ is the standard deviation of the X-ray current and is given by the following equation:

$${I}_{noise} = \sqrt{\frac{1}{N} {\sum }_{i}^{N} {( {I}_{Xray} - {Avg}_{Xray} )}^{2}} \qquad ( 4 )$$

Where I_{Xray} is the X-ray current measured at a particular dose rate.

### EXAMPLE 2-Results and Discussion

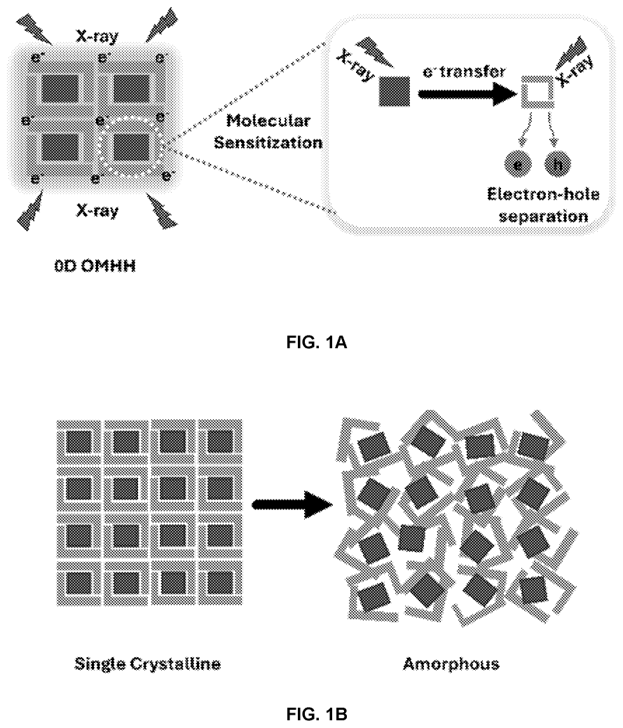

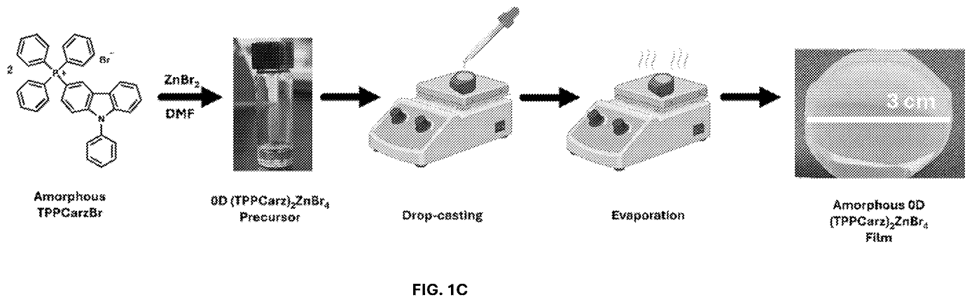

FIG. 1A shows the molecular sensitization scheme in 0D OMMHs, where metal halides act as X-ray absorbers and organic cations as charge transporters. This example describes the development of amorphous 0D OMHH films, rather than single crystals, for direct X-ray detectors, as depicted in FIG. 1B. TPPCarzBr was chosen as the non-crystalline organic salt due to its semiconducting nature, which has been previously demonstrated to form amorphous 0D OMHH films when reacted with MnBr₂ via simple solution processing.

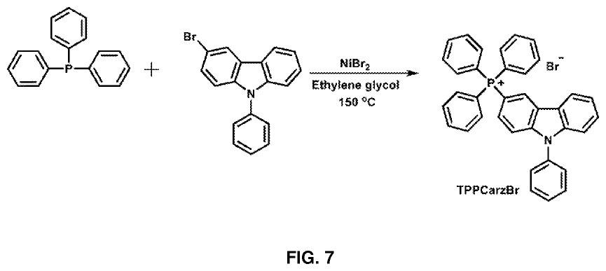

Details of TPPCarzBr synthesis can be found in FIG. 7. By replacing MnBr₂ with the wider-bandgap ZnBr₂ in the reaction with TPPCarzBr, 0D (TPPCarz)₂ZnBr₄ was obtained, allowing for molecular sensitization of TPPCarz⁺ cations by ZnBr₄²⁻ anions. The preparation of amorphous 0D (TPPCarz)₂ZnBr₄ films is illustrated in FIG. 1C. A precursor solution was prepared by dissolving TPPCarzBr and ZnBr₂ in a 2:1 mole ratio at the desired concentrations in dimethylformamide (DMF) solvent at room temperature. The solution was then cast into a resin mold and heated at approximately 150° C. inside a fume hood for two days to remove the solvent as described above. The resulting films, exhibiting excellent uniformity and smoothness, could be easily removed from the mold, with their size, thickness, and area readily adjustable by varying the precursor solution concentration, amount, and mold dimensions.

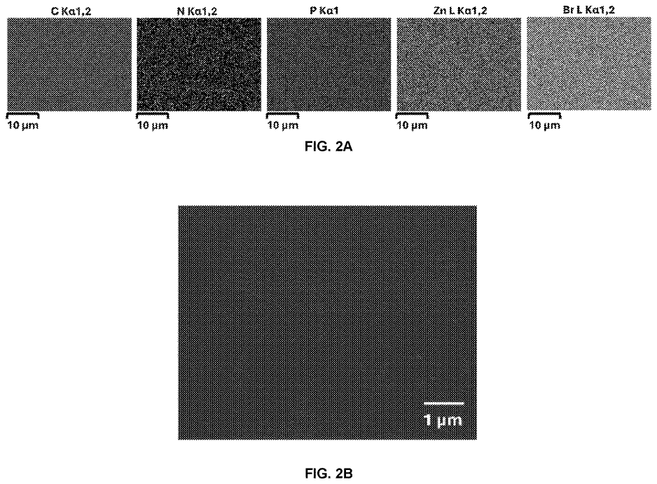

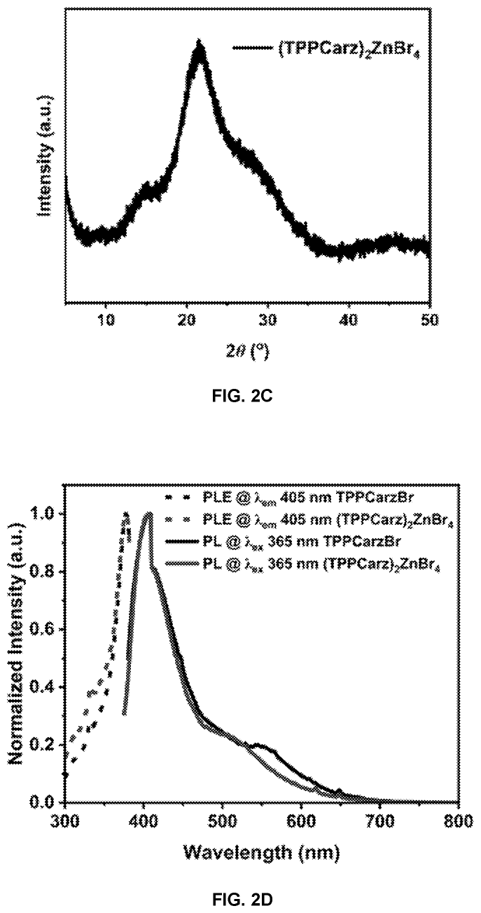

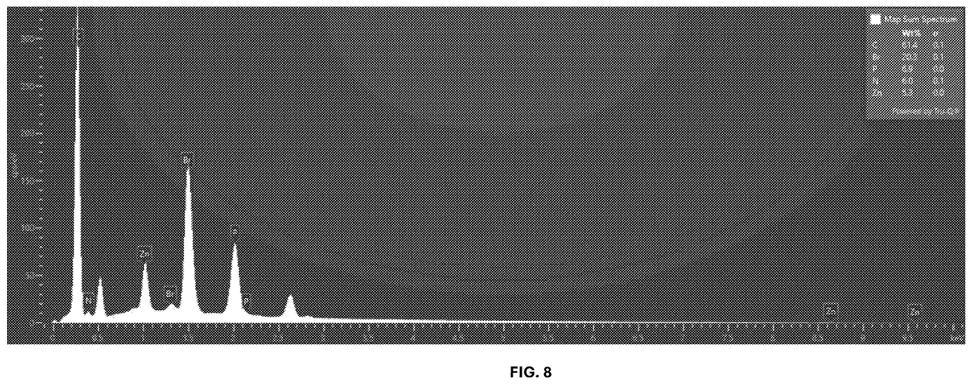

To confirm that the prepared 0D (TPPCarz)₂ZnBr₄ films possessed the desired chemical composition and excellent film morphology, comprehensive compositional, structural, morphological, and photophysical characterizations were conducted. Energy-dispersive X-ray spectroscopy (EDS) analysis, as shown in FIG. 2A, reveals the presence of all expected elements, namely, C, N, P, Zn, and Br, with a uniform spatial distribution throughout the film. The successful formation of 0D (TPPCarz)₂ZnBr₄ through this simple solution processing followed by mild thermal annealing was further verified by the close agreement between the measured elemental composition and the theoretical weight percentages, corresponding to the 2:1 mole ratio of TPPCarzBr and ZnBr₂, as presented in both FIG. 8 and Table 1.

TABLE 1
Percentage weights of theoretical (elemental) analysis
and experimental (EDS) analysis of 0D (TPPCarz)₂ZnBr₄.

| | Elemental Analysis | EDS Analysis | Gained | Lost |
|---|---|---|---|---|
| Element | (%) | (%) | (%) | (%) |
| C | 62.03 | 61.4 | N/A | 0.63 |
| N | 2.01 | 6.0 | 3.99 | N/A |
| P | 4.44 | 6.9 | 2.46 | N/A |
| Zn | 4.69 | 5.3 | 0.61 | N/A |
| Br | 22.92 | 20.3 | N/A | 2.62 |
| H | 3.90 | N/A | | |

| | |
|---|---|
| Gained − Lost | 3.81 |
| (%) | |

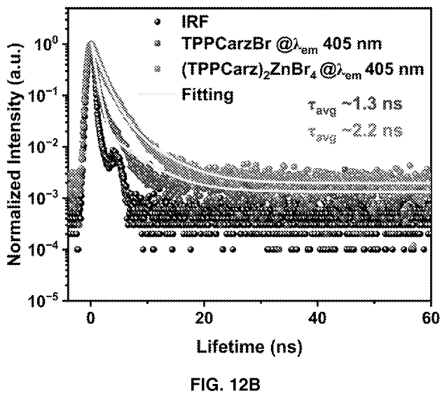

Likewise, to confirm the presence of zinc bromide bonds in 0D (TPPCarz)₂ZnBr₄, the Raman spectra was recorded as shown in FIG. 12A, where Zn—Br stretching vibration could be seen at ~190 cm⁻¹. To evaluate the film's surface smoothness, a scanning electron microscope (SEM) was used. As shown in FIG. 2B, the SEM image exhibits a featureless surface, indicating excellent uniformity and smoothness, which are useful for high-performance detector applications.

The amorphous feature of the solution-processed films was confirmed by using powder X-ray diffraction (PXRD). As shown in FIG. 2C, the PXRD pattern displays distinct broad diffraction features ranging from 2θ=5 to 50°, in contrast to the sharp diffracting peaks observed in crystalline 0D OMHHs, providing clear evidence of the film's intrinsic amorphous nature. With the compositional and structural properties of 0D (TPPCarz)₂ZnBr₄ films characterized, their basic photophysical properties were investigated using steady-state optical spectroscopy. FIG. 2D shows the photoluminescence (PL) emission spectra of solution-processed TPPCarzBr and 0D (TPPCarz)₂ZnBr₄ films, both measured under 365 nm excitation. The deep blue emission of 0D (TPPCarz)₂ZnBr₄ films, peaking at ~405 nm, closely matches that of TPPCarzBr (FIG. 2D), indicating that the emission originates from the lower bandgap TPPCarz⁺ cations, with minimal contribution from the wider bandgap ZnBr₄²⁻ species. Furthermore, time-resolved photoluminescence (TRPL) spectra were recorded for both samples, as shown in FIG. 12B. The short average decay lifetimes of 1.3 and 2.2 ns, respectively, suggest a common emission origin from TPPCarz⁺. These results, in contrast to the green emission of 0D (TPPCarz)₂MnBr₄, originating from MnBr₄²⁻ species with a lower bandgap than TPPCarz⁺ cations, validates the material design strategy. It demonstrates the successful integration of wide-bandgap metal halide anions with smaller-bandgap organic cations, useful for allowing for efficient molecular sensitization of organic cations by metal halides.

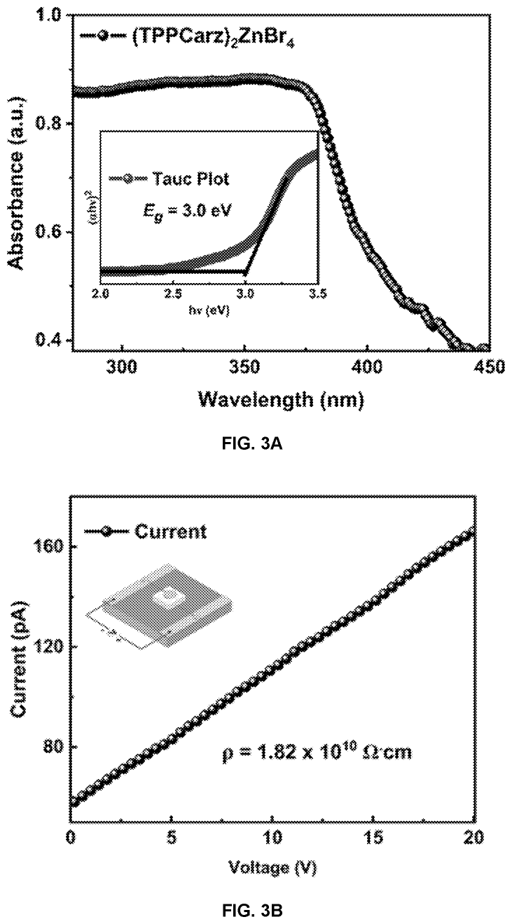

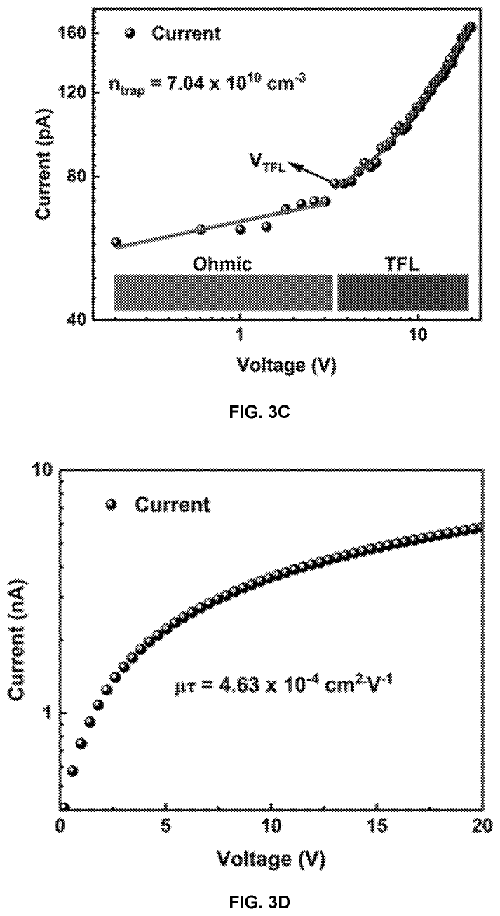

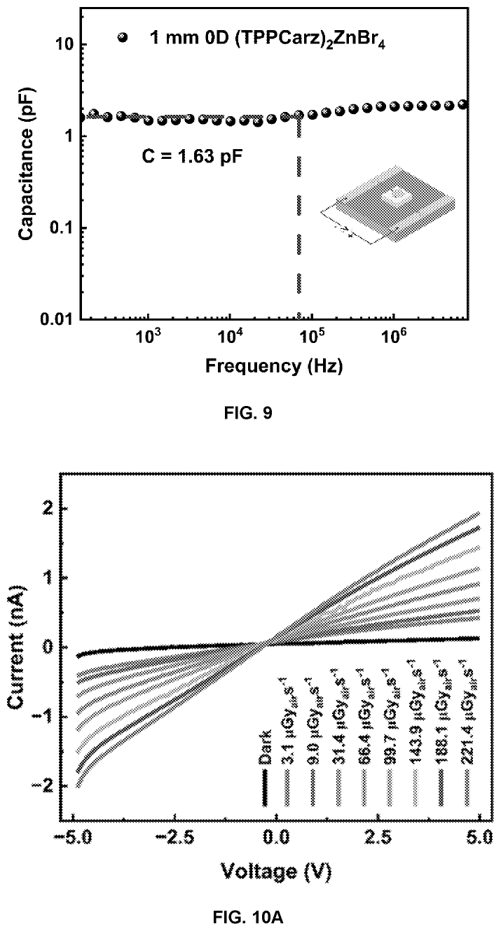

The optical bandgap of amorphous 0D (TPPCarz)₂ZnBr₄ films was estimated from the ultraviolet-visible absorption spectrum, as shown in FIG. 3A. With an absorption edge at ~402 nm, 0D (TPPCarz)₂ZnBr₄ has an optical bandgap of around 3.0 eV, as determined by Tauc plot analysis (inset of FIG. 3A), placing it within the semiconductor range. To characterize the electronic properties of amorphous 0D (TPPCarz)₂ZnBr₄ films, a simple two-terminal vertical device (inset of FIG. 3B) was fabricated using a 1 mm-thick 0D (TPPCarz)₂ZnBr₄ film. The dark bulk resistivity measured as ~1.82×10¹⁰ Ω·cm is comparable to that of molten-grown CdZnTe (3×10¹⁰ Ω·cm) and previously reported 0D lead-free metal halide single crystals (~10¹⁰ Ω·cm), but significantly higher than most of the metal halide perovskite single crystals (~10⁷-10⁹ (cm). Similar to 0D (TPA-P)₂ZnBr₄ single crystals, the high dark resistivity recorded here in 0D (TPPCarz)₂ZnBr₄ films can be attributed to the 0D structure, which has low intrinsic carrier concentrations that effectively suppress dark current and eliminate unfavorable baseline drifting of detectors under real-life operation. Fitting the dark current-voltage (I-V) curve with the space charge-limited current (SCLC) model (FIG. 3C) revealed two distinct regimes, corresponding to I∝Vⁿ: the Ohmic (n=1) and Trap-Filling Limited (TFL, n >2) regimes. The density of trap states, nₜᵣₐₚ of 0D (TPPCarz)₂ZnBr₄ was calculated using Equation (5) below:

$${n}_{trap} = \frac{2 {V}_{TFL} ε {ε}_{0}}{{eL}^{2}} \qquad ( 5 )$$

Where V_{TFL}=3.46 V represents the onset trap-filling voltage (the voltage corresponding to the regime where all the available trap states are filled with injected carriers from the metal electrodes), ε is the permittivity estimated from the Capacitance vs. Frequency plot (shown in FIG. 9), ε₀=the vacuum permittivity (8.854×10⁻¹² F·m⁻¹), e=elementary charge (1.602×10⁻¹⁹ C), and L=the thickness of the film sample (1 mm). Using these parameters, the nₜᵣₐₚ value of solution-processed amorphous 0D (TPPCarz)₂ZnBr₄ films was determined to be 7.04×10¹⁰ cm⁻³, a remarkably low value.

Without wishing to be bound by any theory, this suggests that the film has a defect-free nature, which is highly advantageous for minimizing charge carrier recombination and enhancing carrier extraction efficiency. Indeed, this trap density is significantly lower than that of many commercially available inorganic semiconductors, including polycrystalline Si (10¹³-10¹⁴ cm⁻³) and CdTe/CdS (10¹¹-10¹³ cm⁻³), as well as pure crystalline organic semiconductors like pentacene (10¹⁴-10¹⁵ cm⁻³) and rubrene (~10¹⁶ cm⁻³). To evaluate the charge carrier collection efficiency of 0D (TPPCarz)₂ZnBr₄ films for direct X-ray detection, the charge carrier mobility lifetime product (μτ) was measured under a continuous X-ray irradiation of 221.39 μGyₐᵢᵣ s⁻¹. As shown in FIG. 3D, the voltage-dependent photoconductivity ut was estimated to be 4.63×10⁻⁴ cm²·V⁻¹ using the Modified Hecht equation (Equation (6) below):

$$I = \frac{{I}_{0} μ τ V}{{L}^{2}} [ 1 - exp ( \frac{- {L}^{2}}{μ τ V} ) ] \qquad ( 6 )$$

Where L is the thickness of the 0D (TPPCarz)₂ZnBr₄ film sample (1 mm) sandwiched between two vertical silver electrodes with a 1×1 mm² area, and I₀ represents the saturated photocurrent reached under a 20 V bias voltage. The high-mobility lifetime product of 0D (TPPCarz)₂ZnBr₄ films can be attributed to the presence of the charge-conducting organic cations, TPPCarz⁺. This value is comparable to that of commercially available inorganic CdZnTe (~10⁻³-10⁻⁵ cm²·V⁻¹) and CsPbBr₃ detectors (~10⁻³ cm²·V⁻¹), and much higher than that of α-Se detectors (10⁻⁷ cm²·V⁻¹).

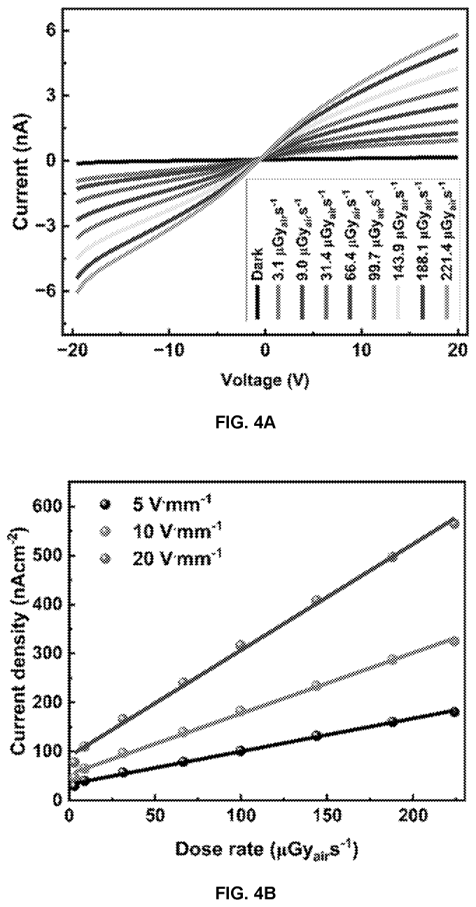

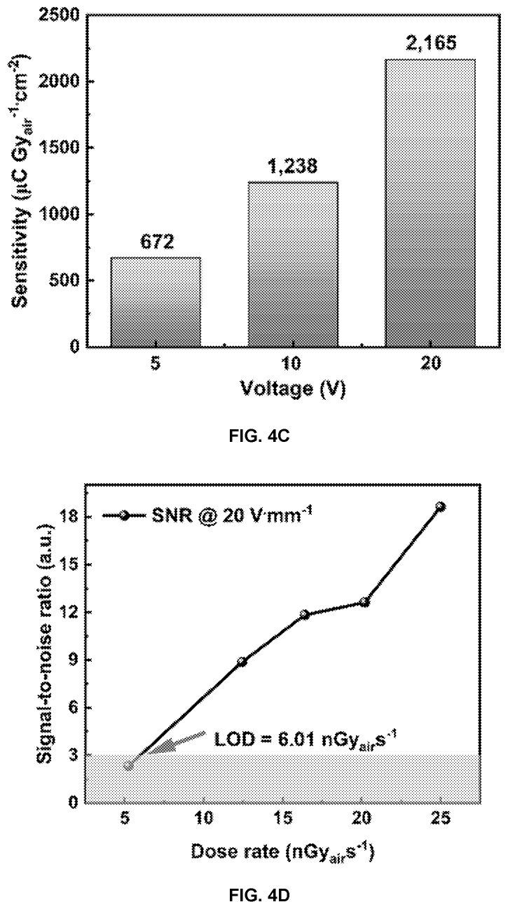

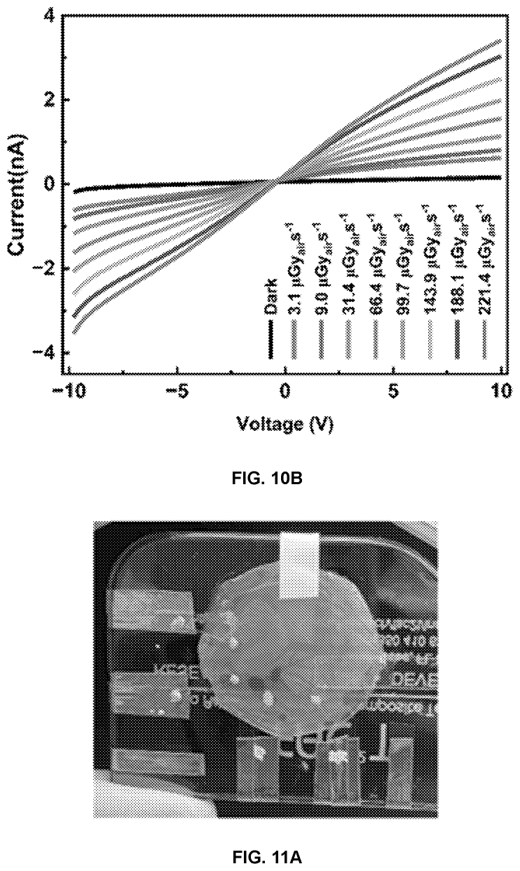

The useful electrical properties of solution-processed amorphous 0D (TPPCarz)₂ZnBr₄ films, including high bulk resistivity, low density of trap states, and a large ut, make them promising semiconductors for direct X-ray detectors. To further evaluate its capabilities, direct X-ray detectors were fabricated based on 0D (TPPCarz)₂ZnBr₄ films and their performance was tested at various electric fields and X-ray doses inside a homemade dark box. A tungsten anode X-ray tube with a maximum voltage of 40 kV and a current of 100 μA was used as the radiation source. Under an applied electric field of 20 V·mm⁻¹ in ambient air, the effect of increasing the X-ray dose rate on the measured photocurrent was investigated. As shown in FIG. 4A, the I-V plot reveals a photocurrent increase at 20 V·mm⁻¹ when transitioning from dark conditions to an X-ray dose of 3.1 μGyₐᵢᵣs⁻¹. Results for similar I-V measurements at 5 V·mm⁻¹ and 10 V·mm⁻¹ are provided in FIGS. 10A-10B, respectively. Moreover, FIG. 4A demonstrates that 0D (TPPCarz)₂ZnBr₄ detectors exhibited exceptional operational stability, with a consistent increase in photocurrent as the X-ray tube current was adjusted from 20 μA to 100 μA, reaching a maximum X-ray dose of 221.39 μGyₐᵢᵣs⁻¹. Like in X-ray detectors based on 0D (TPA-P)₂ZnBr₄ single crystals, a well-suppressed ionic migration was achieved in devices based on amorphous 0D (TPPCarz)₂ZnBr₄ films. Without wishing to be bound by any theory, this could be attributed to their 0D structure, where the bulky TPPCarz⁺ cations introduce steric effects that increase the activation energy barrier for ionic migration.

The sensitivity and limit of detection are two other useful performance metrics of X-ray detectors, particularly in medical applications. The photocurrent was measured under varying electric fields and increasing X-ray dose rates to determine the sensitivity of amorphous 0D (TPPCarz)₂ZnBr₄ film-based detectors. As expected for a useful X-ray detector, FIG. 4B displays a linear relationship between the generated photocurrent densities and the X-ray dose rates across all tested electric fields. The sensitivity(S) was extracted from the plots of current density (J_{X-ray} and J_{dark} represent photocurrent and dark current densities) versus X-ray dose rate (D) at different voltages using Equation (7) below:

$$S = \frac{( {J}_{X - ray} - {J}_{dark} )}{D} \qquad ( 7 )$$

As shown in FIG. 4C, the sensitivity of amorphous 0D (TPPCarz)₂ZnBr₄ film-based detectors was measured at different electric fields, yielding 672 μC Gyₐᵢᵣ⁻¹ cm⁻² at 5 V·mm⁻¹ and increasing to 1,238 μC Gyₐᵢᵣ⁻¹ cm⁻² at 10 V·mm⁻¹. A high sensitivity of 2, 165 μC Gyₐᵢᵣ⁻¹ cm⁻² was recorded at 20 V·mm⁻¹, outperforming commercially amorphous α-Se-based detectors (440 μC Gyₐᵢᵣ⁻¹ cm⁻²), which requires a much higher operating electric field of 15, 000 V·mm⁻¹.

The amorphous 0D (TPPCarz)₂ZnBr₄ film-based detectors operated at a field of 20 V·mm⁻¹ also exhibit improved performance over several reported lead-free bismuth-based materials, including 3D Cs₂AgBiBr₆ single crystals (1,974 μC Gyₐᵢᵣ⁻¹ cm⁻² at 50 V·mm⁻¹), 0D (R-PPA) 2BiI₅ single crystals (150 μC Gyₐᵢᵣ⁻¹ cm⁻² at 10 V·mm⁻¹), 0D MA₃Bi₂I₉ single crystals (1,947 μC Gyₐᵢᵣ⁻¹ cm⁻² at 60 V·mm⁻¹), and comparable to the 0D AG₃Bi₂I₉ polycrystalline wafers (2,675 μC Gyₐᵢᵣ⁻¹ cm⁻² at 20 V·mm⁻¹). The X-ray detection limit (LOD) of amorphous 0D (TPPCarz)₂ZnBr₄ film-based detectors at 20 V mm⁻¹ was determined to be 6.01 nGyₐᵢᵣs⁻¹, as shown in FIG. 4D, where LOD is the equivalent X-ray dose rate corresponding to a signal-to-noise ratio (SNR) of 3. This low LOD is the best reported to date for 0D OMHHs-based X-ray detectors. This LOD value is more than 900 times lower than the X-ray dose rate of 5.5 μGyₐᵢᵣs⁻¹ used for standard medical diagnostics, highlighting the use of the amorphous 0D (TPPCarz)₂ZnBr₄ films for safe and high-performance X-ray detectors in medical applications. Table 2 summarizes the performance of devices based on the amorphous 0D (TPPCarz)₂ZnBr₄ films in comparison with existing amorphous materials, including α-Se and other organic-inorganic hybrid films.

TABLE 2
Comparison of the performance of various amorphous materials used in direct X-ray detectors.

| | | | | Voltage | | | | | |
|---|---|---|---|---|---|---|---|---|---|
| | Sensitivity | | Limit of | (V)/Electric | | | | | |
| | (μC | Dark | Detection | Field | μr | Resistivity | | | |
| Material | Gy⁻¹cm⁻²) | Current | (nGy⁻¹s⁻¹) | (Vmm⁻¹) | (cm²V⁻¹) | (Ωcmr⁻¹) | Thick. | | Year |
| α-Se | 440 | — | 5500 | 15,000 | 1 × 10⁻¹ | — | — | | 2000 |
| | | | | Vmm⁻¹ | | | | | |
| Ga₂O₃ | 0.27 | ~400 nA | | 100 V | | | 250 | nm | 2018 |
| 0D | 2193 | ~110 PA | 19.69 | 50 V | 5.4 × 10⁻⁵ | 6.94 × 10¹² | 0.5 | mm | 2024 |
| MTP₃Bi₂X₀ | | | | | | | | | |
| 0D | 2165 | ~100 pA | 6.01 | 20 V | 4.63 × 10⁻⁴ | 1.82 × 10¹⁰ | 1.0 | mm | This |
| (TPPCarz)₂ZnBr₄ | | | | | | | | | work |

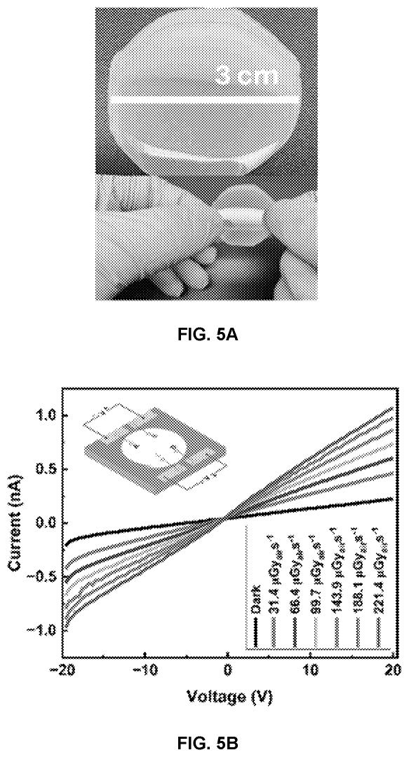

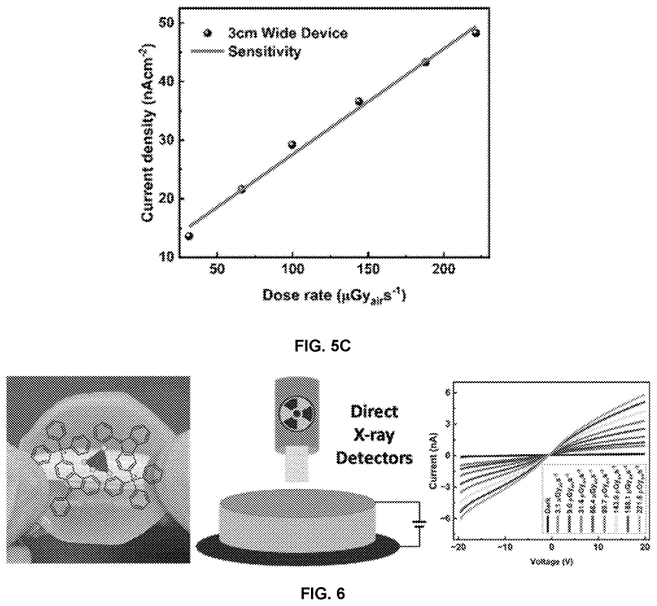

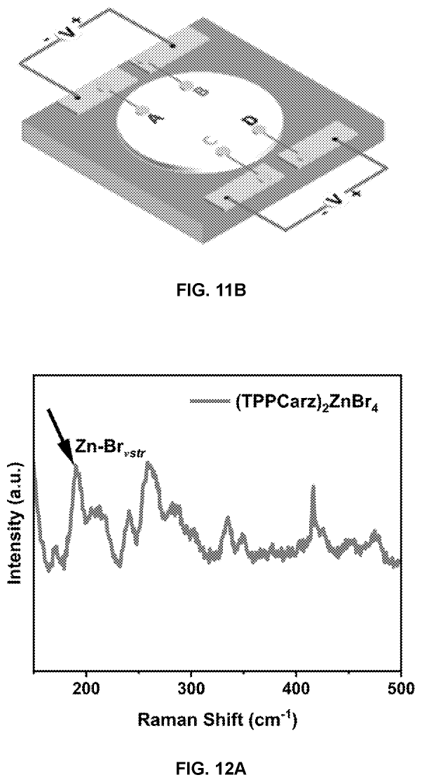

To further demonstrate the feasibility of fabricating large-area X-ray detectors using solution-processed amorphous 0D (TPPCarz)₂ZnBr₄ films, a larger film that is about 30 times larger than the 1 mm×1 mm film was prepared, and approximately 3 cm in diameter (FIG. 5A) with a thickness of ~1 mm (FIG. 5B) using a larger mold. X-ray detectors were then fabricated using a lateral device architecture with silver electrodes (FIGS. 11A-11B). The performance of large-area detectors was evaluated under similar test conditions as before, applying an electric field of 20 V·mm⁻¹ and X-ray dose rates up to 221.39 μGyₐᵢᵣs⁻¹. As shown in the I-V plot in FIG. 5B, the detector exhibited stable performance and a steady increase in the measured photocurrent across the entire range of X-ray dose rates applied. Remarkably, the 3 cm wide detector achieved a sensitivity of 1,802 μC Gyₐᵢᵣ⁻¹ cm⁻² at 20 V·mm⁻¹ (FIG. 5C), which suggests that the 0D (TPPCarz)₂ZnBr₄ film can be used for larger area detector applications.

In summary, high-quality, large-area amorphous 0D (TPPCarz)₂ZnBr₄ films were developed using a facile solution-processing technique for the first time. These films exhibited useful electronic properties, including a large bulk resistivity of 1.82×10¹⁰ Ω·cm, a high ut of 4.63×10⁻⁴ cm²·V⁻¹, an impressive sensitivity of 2,165 μC Gyₐᵢᵣ⁻¹ cm⁻², and an ultra-low detection limit of 6.01 nGyₐᵢᵣs⁻¹, representing the best performance reported to date for solution-processed amorphous materials-based X-ray detectors. Moreover, 0D (TPPCarz)₂ZnBr₄ films can be readily processed into large-area (~3 cm diameter) flat detector films and seamlessly integrated with external readout circuitry for high-performance X-ray detection in ambient air. This work highlights the tunability of 0D OMHHs, particularly their facile solution processibility, versatility, and unique material properties, making them useful for next-generation large-area flat panel detectors.

### Exemplary Aspects

Example 1. A direct X-ray detector comprising: an amorphous organic metal halide hybrid (OMHH) material having a composition [R]_{z}(MₘXₙ)^{z-}, wherein (MₘXₙ)^{z-} is a metal halide anion, R⁺ is an organic cation, and wherein z, m, and n are integers; and at least one electrode in electrical communication with the OMHH material.

Example 2. The direct X-ray detector of any one of the examples herein, particularly Example 1, wherein z is 1 to 6, m is 1 to 12, and n is 1 to 24.

Example 3. The direct X-ray detector of any one of the examples herein, particularly Example 1 or 2, wherein the OMHH is a thin film.

Example 4. The direct X-ray detector of any one of the examples herein, particularly Examples 1-3, wherein M is a metal selected from Mn, Ni, Cu, Zn, Pd, Ag, Sn, Sb, Ti, Pb, and Bi, and X is a halide chosen from Cl, Br, I, and combinations thereof.

Example 5. The direct X-ray detector of any one of the examples herein, particularly Examples 1-4, wherein R⁺ comprises

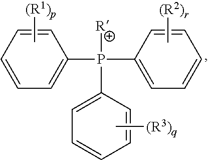

wherein R′ is a substituted or unsubstituted light-emitting organic unit covalently bound to phosphorus; wherein R¹, R², and R³, independently are selected at each occurrence from F, Cl, Br, I, NO₂, CN, COOH, C₁₋₈alkyl, C₁₋₈heteroalkyl, C₆₋₁₄aryl, C₃₋₈cycloalkyl, 3- to 8-membered heterocyclyl, and 5- to 8-membered heteroaryl; and wherein p, q, and r are independently integer from 0 to 5.

Example 6. The direct X-ray detector of any one of the examples herein, particularly Example 5, wherein R′ comprises

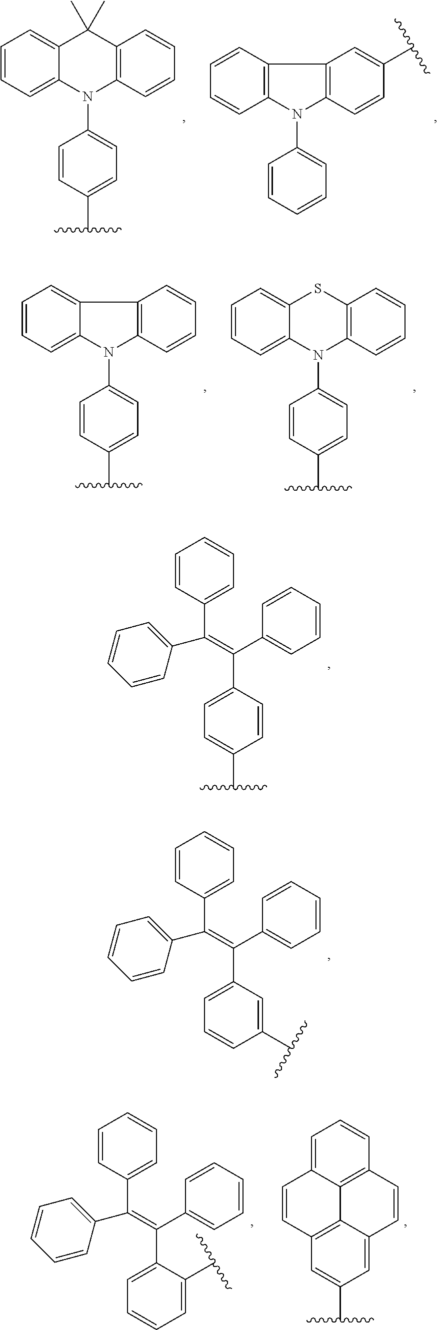

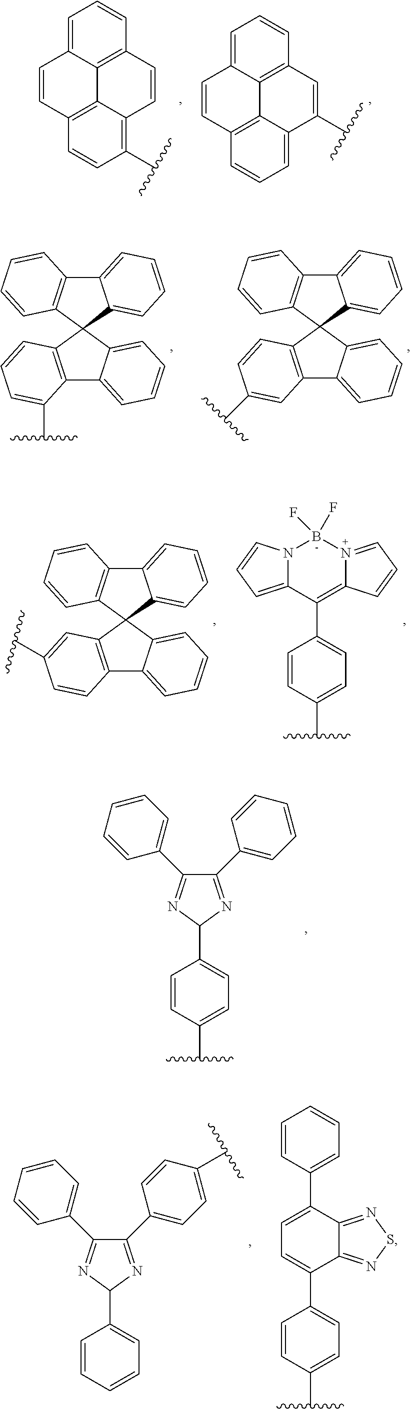

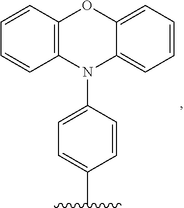 [SMILES: *c1ccc(N2c3ccccc3Oc3ccccc32)cc1]

or a combination thereof.

Example 7. The direct X-ray detector of any one of the examples herein, particularly Examples 1-6, wherein R⁺ is selected from triphenyl (9-phenyl-9H-carbazol-3-yl)phosphonium (TPPCarz⁺), (4-(9,9-dimethylacridin-10 (9H)-yl)phenyl) triphenylphosphonium (DMAC-TPP⁺), 4-(10H-phenoxazin-10-yl)phenyltriphenylphosphonium (PXZ-TPP⁺), or any combination thereof.

Example 8. The direct X-ray detector of any one of the examples herein, particularly Examples 1-7, wherein R⁺ has a bandgap that is lower than or equal to (MₘXₙ)_{z}⁻ such that the metal halide anions are configured to absorb X-ray radiation primarily and the R⁺ is configured to transport charge carriers generated upon absorption of the X-ray radiation.

Example 9. The direct X-ray detector of any one of the examples herein, particularly Examples 1-8, wherein the OMHH is (TPPCarz)₂ZnBr₄, or (DMAC-TPP)₂ZnBr₄, or (PXZ-TPP)₂ZnBr₄.

Example 10. The direct X-ray detector of any one of the examples herein, particularly Examples 1-9, wherein the OMHH exhibits a bulk resistivity greater than 10¹⁰ Ω·cm.

Example 11. The direct X-ray detector of any one of the examples herein, particularly Examples 1-10, wherein the OMHH exhibits a bulk resistivity of at least 10¹⁰ Ω·cm.

Example 12. The direct X-ray detector of any one of the examples herein, particularly Examples 1-11, wherein the OMHH exhibits a trap density of less than 10¹¹ cm⁻³.

Example 13. The direct X-ray detector of any one of the examples herein, particularly Examples 1-12, wherein the OMHH exhibits a charge carrier mobility-lifetime product of at least about 10⁻⁴ cm²·V⁻¹

Example 14. The direct X-ray detector of any one of the examples herein, particularly Examples 1-13, wherein the direct X-ray detector exhibits a sensitivity of at least about 1,500 μC Gyₐᵢᵣ⁻¹ cm⁻² at 20 V·mm⁻¹.

Example 15. The direct X-ray detector of any one of the examples herein, particularly Examples 1-14, wherein the direct X-ray detector exhibits a sensitivity of at least about 2,000 μC Gyₐᵢᵣ⁻¹ cm⁻² at 20 V mm⁻¹.

Example 16. The direct X-ray detector of any one of the examples herein, particularly Examples 1-15, wherein the direct X-ray detector has a detection limit of less than or equal to 10 nGyₐᵢᵣs⁻¹ at 20 V·mm⁻¹.

Example 17. The direct X-ray detector of any one of the examples herein, particularly Examples 1-16, wherein ionic migration is substantially suppressed.

Example 18. The direct X-ray detector of any one of the examples herein, particularly Examples 1-17, wherein the detector comprises a first electrode and a second electrode, and wherein the amorphous OMHH material is disposed between the first and the second electrodes.

Example 19. The direct X-ray detector of any one of the examples herein, particularly Example 18, wherein the first and the second electrodes comprise Au, Ag, indium tin oxide (ITO), carbon nanotubes (CNT), Cu, Ni, Al, or combinations thereof.

Example 20. The direct X-ray detector of any one of the examples herein, particularly Examples 1-19, wherein the detector has at least one lateral dimension that is 1 to 50 cm.

Example 21. The direct X-ray detector of any one of the examples herein, particularly Example 20, wherein the at least one lateral dimension is a diameter.

Example 22. The direct X-ray detector of any one of the examples herein, particularly Example 21, wherein the at least one dimension is a width of the detector.

Example 23. The direct X-ray detector of any one of the examples herein, particularly Examples 2-22, wherein the film has a thickness of 0.1 mm⁻¹⁰ mm.

Example 24 The direct X-ray detector of any one of the examples herein, particularly Examples 1-23, wherein the detector exhibits stable performance of reduction of less than 2% in measured photocurrent at X-ray dose rates up to 221.39 μGyₐᵢᵣs⁻¹ at 20 V·mm⁻¹ for 24 h.

Example 25. The direct X-ray detector of any one of the examples herein, particularly Examples 1-24, wherein the amorphous material is formed by a solution-based process.

Example 26. The direct X-ray detector of any one of the examples herein, particularly Examples 1-25, wherein the amorphous OMHH material is formed at a temperature of less than 200° C.

Example 27. A method of detecting X-ray radiation, the method comprising: exposing an amorphous organic metal halide hybrid (OMHH) material having a composition [R]_{z}(MₘXₙ)^{z-} to X-ray radiation, wherein (MₘXₙ)^{z-} is a metal halide anion, R⁺ is an organic cation, and wherein z, m, and n are integers, wherein the metal halide anions configured to absorb X-ray radiation and the R⁺ is configured to transport charge carriers generated upon absorption of the X-ray radiation, applying an electric field across the material; and detecting a signal generated by the transported charge carriers.

Example 28. The method of any one of the examples herein, particularly Example 27, wherein z is 1 to 6, m is 1 to 12, and n is 1 to 24.

Example 29. The method of any one of the examples herein, particularly Example 27-28, wherein the OMHH is a thin film.

Example 30. The method of any one of the examples herein, particularly Examples 27-29, wherein M is a metal selected from Mn, Ni, Cu, Zn, Pd, Ag, Sn, Sb, Ti, Pb, and Bi, and X is a halide chosen from Cl, Br, I, and combinations thereof.

Example 31. The method of any one of the examples herein, particularly Examples 27-30, wherein R⁺ comprises

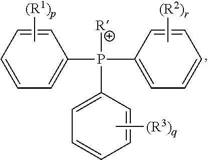

wherein R′ is a substituted or unsubstituted light-emitting organic unit covalently bound to phosphorus; wherein R¹, R², and R³, independently are selected at each occurrence from F, Cl, Br, I, NO₂, CN, COOH, C₁₋₈alkyl, C₁₋₈heteroalkyl, C₆₋₁₄aryl, C₃₋₈cycloalkyl, 3- to 8-membered heterocyclyl, and 5- to 8-membered heteroaryl; and wherein p, q, and r are independently integer from 0 to 5.

Example 32. The method of any one of the examples herein, particularly Example 31, wherein R′ comprises

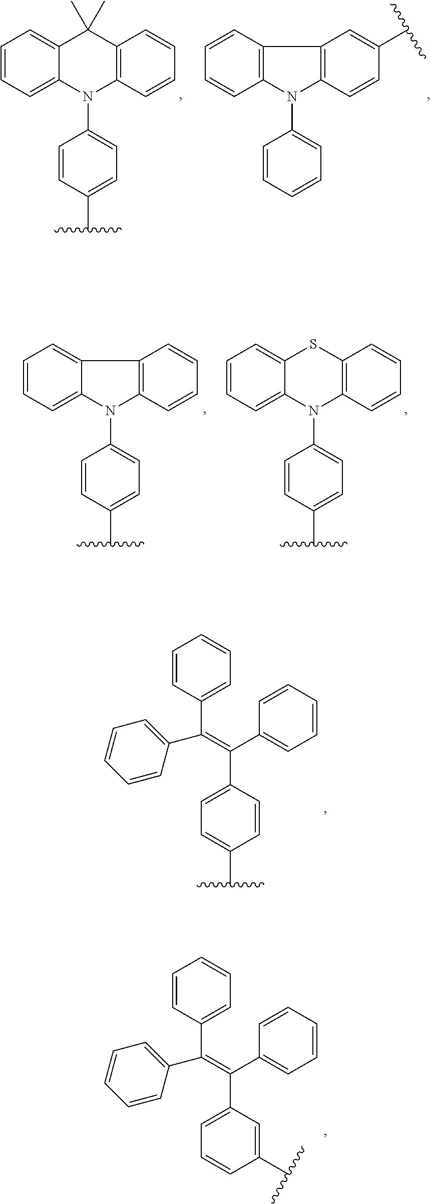

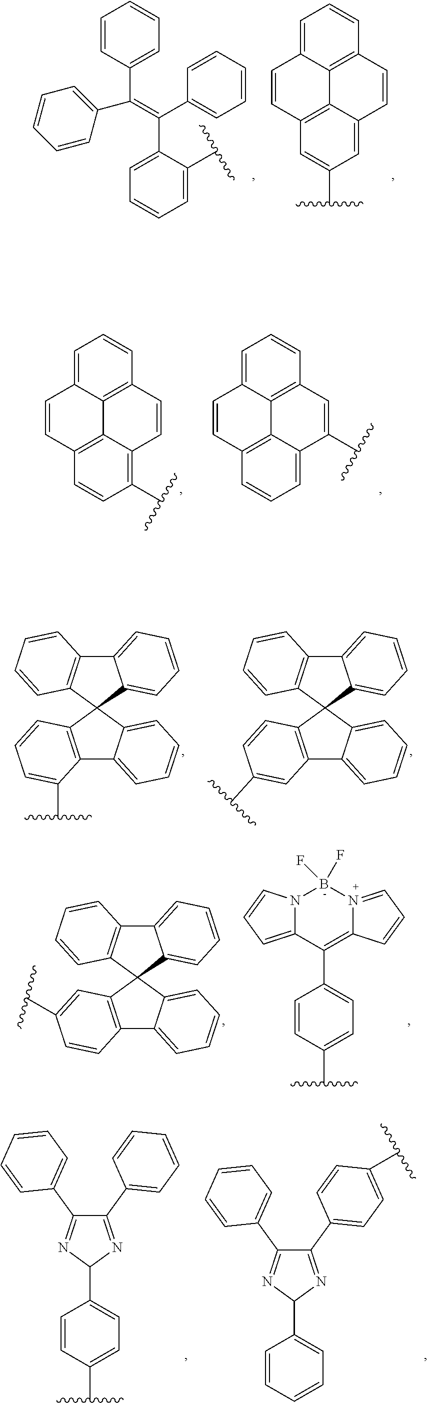

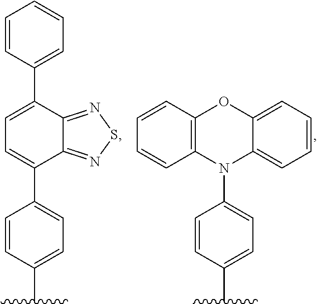

or a combination thereof.

Example 33. The method of any one of the examples herein, particularly Examples 27-32, wherein R⁺ is selected from triphenyl (9-phenyl-9H-carbazol-3-yl)phosphonium (TPPCarz⁺), (4-(9,9-dimethylacridin-10 (9H)-yl)phenyl) triphenylphosphonium (DMAC-TPP⁺), (4-(10H-phenoxazin-10-yl)phenyltriphenylphosphonium (PXZ-TPP⁺), or any combination thereof.

Example 34. The method of any one of the examples herein, particularly Examples 27-33, wherein R⁺ has a bandgap that is lower than or equal to (MₘXₙ)_{z}⁻ such that the metal halide anions are configured to absorb X-ray radiation primarily and the R⁺ is configured to transport charge carriers generated upon absorption of the X-ray radiation.

Example 35. The method of any one of the examples herein, particularly Examples 27-34, wherein the OMHH is (TPPCarz)₂ZnBr₄, or (DMAC-TPP)₂ZnBr₄, or (PXZ-TPP)₂ZnBr₄.

Example 36. The method of any one of the examples herein, particularly Examples 27-35, wherein the OMHH exhibits a bulk resistivity greater than 10¹⁰ Ω·cm.

Example 37. The method of any one of the examples herein, particularly Examples 27-36, wherein the OMHH exhibits a bulk resistivity of at least 10¹⁰ Ω·cm.

Example 38. The method of any one of the examples herein, particularly Examples 27-37, wherein the OMHH exhibits a trap density of less than 10¹¹ cm⁻³.

Example 39. The method of any one of the examples herein, particularly Examples 27-38, wherein the OMHH exhibits a charge carrier mobility-lifetime product of at least about 10⁻⁴ cm²·V⁻¹

Example 40. The method of any one of the examples herein, particularly Examples 27-39, wherein the direct X-ray detector exhibits a sensitivity of at least about 1,500 UC Gyₐᵢᵣ⁻¹ cm⁻² at 20 V·mm⁻¹.

Example 41. The method of any one of the examples herein, particularly Examples 27-40, wherein the direct X-ray detector exhibits a sensitivity of at least about 2,000 μC Gyₐᵢᵣ⁻¹ cm⁻² at 20 V mm⁻¹.

Example 42. The method of any one of the examples herein, particularly Examples 27-41, wherein the direct X-ray detector has a detection limit of less than or equal to 10 nGyₐᵢᵣs⁻¹ at 20 V·mm⁻¹.

Example 43. The method of any one of the examples herein, particularly Examples 27-42, wherein ionic migration is substantially suppressed.

Example 44. The method of any one of the examples herein, particularly Examples 27-43, wherein the detector comprises a first electrode and a second electrode, and wherein the amorphous OMHH material is disposed between the first and the second electrodes.

Example 45. The method of any one of the examples herein, particularly Example 44, wherein the first and the second electrodes comprise Au, Ag, indium tin oxide (ITO), carbon nanotubes (CNT), Cu, Ni, Al, or combinations thereof.

Example 46 The method of any one of the examples herein, particularly Examples 27-45, wherein the detector has at least one lateral dimension that is 1 to 50 cm.

Example 47. The method of any one of the examples herein, particularly Example 46, wherein the at least one lateral dimension is a diameter.

Example 48. The method of any one of the examples herein, particularly Example 46, wherein the at least one dimension is a width of the detector.

Example 49. The method of any one of the examples herein, particularly Examples 29-48, wherein the film has a thickness of 0.1 mm⁻¹⁰ mm.

Example 50. The method of any one of the examples herein, particularly Examples 27-49, wherein the detector exhibits stable performance of reduction of less than 2% in measured photocurrent at X-ray dose rates up to 221.39 μGyₐᵢᵣs⁻¹ at 20 V·mm⁻¹ for 24 h.

Example 51. The method of any one of the examples herein, particularly Examples 27-50, wherein the amorphous material is formed by a solution-based process.

Example 52. The method of any one of the examples herein, particularly Examples 27-51, wherein the amorphous OMHH material is formed at a temperature of less than 200° C.

Example 53. A method of fabricating an X-ray detector, the method comprising: disposing a zero-dimensional amorphous organic metal halide hybrid (OMHH) film having a composition [R]_{z}(MₘXₙ)^{z-} between a first electrode and a second electrode, wherein (MₘXₙ)^{z-} is a metal halide anion, R⁺ is an organic cation, and wherein z, m, and n are integers.

Example 54. The method of any one of the examples herein, particularly Example 53, wherein the amorphous OMHH film is formed by: preparing a precursor solution comprising an organic halide salt and a metal halide salt dissolved in a solvent; depositing the precursor solution; and annealing the deposited precursor solution.

1. A direct X-ray detector comprising:
an amorphous organic metal halide hybrid (OMHH) material having a composition [R]_{z}(MₘXₙ)^{z-}, wherein (MₘXₙ)^{z-} is a metal halide anion, R⁺ is an organic cation, and wherein z, m, and n are integers; and at least one electrode in electrical communication with the OMHH material.

2. The direct X-ray detector of claim 1, wherein the OMHH is a thin film.

3. The direct X-ray detector of claim 1, wherein M is a metal selected from Mn, Ni, Cu, Zn, Pd, Ag, Sn, Sb, Ti, Pb, and Bi, and X is a halide chosen from Cl, Br, I, and combinations thereof.

4. The direct X-ray detector of claim 1, wherein R⁺ comprises 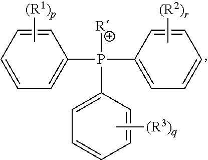
wherein R′ is a substituted or unsubstituted light-emitting organic unit covalently bound to phosphorus;
wherein R¹, R², and R³, independently are selected at each occurrence from F, Cl, Br, I, NO₂, CN, COOH, C₁₋₈alkyl, C₁₋₈heteroalkyl, C₆₋₁₄aryl, C₃₋₈cycloalkyl, 3- to 8-membered heterocyclyl, and 5- to 8-membered heteroaryl; and
p, q, and r are independently integer from 0 to 5.

5. The direct X-ray detector of claim 4, wherein R′ comprises 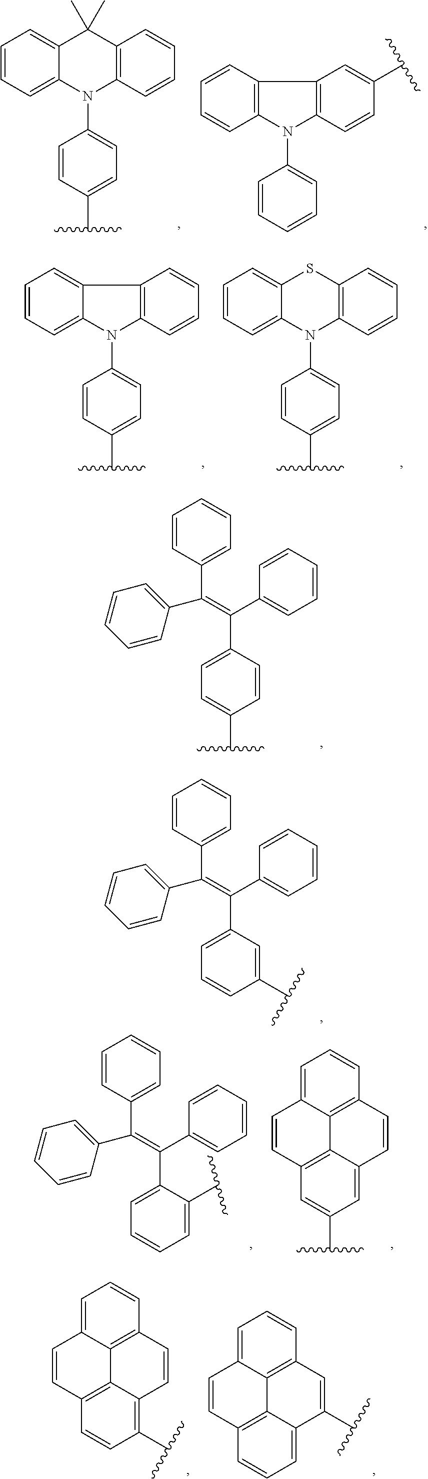 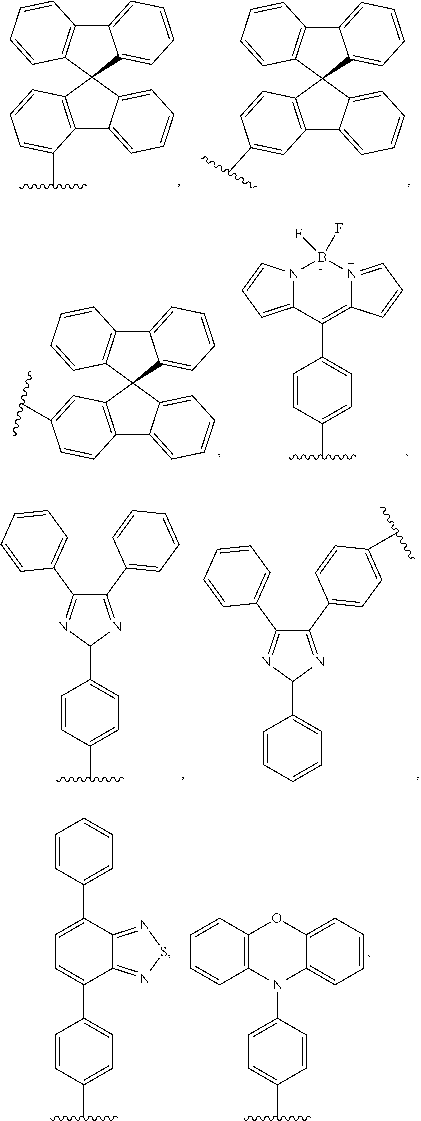
or a combination thereof.

6. The direct X-ray detector of claim 5, wherein R⁺ is selected from triphenyl (9-phenyl-9H-carbazol-3-yl)phosphonium (TPPCarz⁺), (4-(9,9-dimethylacridin-10 (9H)-yl)phenyl) triphenylphosphonium (DMAC-TPP⁺), (4-(10H-phenoxazin-10-yl)phenyltriphenylphosphonium (PXZ-TPP⁺), or any combination thereof.

7. The direct X-ray detector of claim 1, wherein R⁺ has a bandgap that is lower than or equal to (MₘXₙ)^{z-} such that the metal halide anions are configured to absorb X-ray radiation primarily and the R⁺ is configured to transport charge carriers generated upon absorption of the X-ray radiation.

8. The direct X-ray detector of claim 6, wherein the OMHH is (TPPCarz)₂ZnBr₄, or (DMAC-TPP)₂ZnBr₄, or (PXZ-TPP)₂ZnBr₄.

9. The direct X-ray detector of claim 1, wherein the OMHH exhibits a bulk resistivity greater than 10¹⁰ Ω·cm.

10. The direct X-ray detector of claim 1, wherein the OMHH exhibits a trap density of less than 10¹¹ cm⁻³.

11. The direct X-ray detector of claim 1, wherein the OMHH exhibits a charge carrier mobility-lifetime product of at least about 10⁻⁴ cm²·V⁻¹.

12. The direct X-ray detector of claim 1, wherein the direct X-ray detector exhibits a sensitivity of at least about 1,500 C Gyₐᵢᵣ⁻¹ cm⁻² at 20 V·mm⁻¹.

13. The direct X-ray detector of claim 1, wherein the direct X-ray detector has a detection limit of less than or equal to 10 nGyₐᵢᵣ s⁻¹ at 20 V·mm⁻¹.

14. The direct X-ray detector of claim 1, wherein the detector comprises a first electrode and a second electrode, and wherein the amorphous OMHH material is disposed between the first and the second electrodes.

15. The direct X-ray detector of claim 14, wherein the first and the second electrodes comprise Au, Ag, indium tin oxide (ITO), carbon nanotubes (CNT), Cu, Ni, Al, or combinations thereof.

16. The direct X-ray detector of claim 2, wherein the film has a thickness of 0.1 mm⁻¹⁰ mm.

17. The direct X-ray detector of claim 1, wherein the detector exhibits stable performance of reduction of less than 2% in measured photocurrent at X-ray dose rates up to 221.39 μGyₐᵢᵣs⁻¹ at 20 V·mm⁻¹ for 24 h.

18. The direct X-ray detector of claim 1, wherein the amorphous material is formed by a solution-based process at a temperature of less than 200° C.

19. A method of detecting X-ray radiation, the method comprising:
exposing an amorphous organic metal halide hybrid (OMHH) material having a composition [R]_{z}(MₘXₙ)^{z-} to X-ray radiation,
wherein (MₘXₙ)-2 is a metal halide anion, R⁺ is an organic cation, and wherein z, m, and n are integers, wherein the metal halide anions configured to absorb X-ray radiation and the R⁺ is configured to transport charge carriers generated upon absorption of the X-ray radiation
applying an electric field across the material; and
detecting a signal generated by the transported charge carriers.

20. A method of fabricating an X-ray detector, the method comprising:
disposing a zero-dimensional amorphous organic metal halide hybrid (OMHH) film having a composition [R]_{z}(MₘXₙ)^{z-} between a first electrode and a second electrode, wherein (MₘXₙ)^{z-} is a metal halide anion, R⁺ is an organic cation, and wherein z, m, and n are integers.
